(19) United States
(12) Patent Application Publication
(10) Pub. No.: US 2007/0141570 A1
(43) Pub. Date: Jun. 21, 2007

(54) Association of polymorphic kinase anchor proteins with cardiac phenotypes and related methods

(75) Inventors: Andreas Braun, San Diego, CA (US); Stefan M. Kammerer, San Diego, CA (US)

Correspondence Address: GRANT ANDERSON LLP;C/O PORTFOLIOIP, PO BOX 52050, MINNEAPOLIS, MN, 55402, US

(73) Assignee: Sequenom, Inc., San Diego, CA (US)

(21) Appl. No.: 10/548,336

(22) PCT Filed: Mar. 5, 2004
(86) PCT No.: PCT/US04/06740

§ 371 Date: Sep. 9, 2006

Related U.S. Application Data
(60) Provisional application No. 60/453,350, filed on Mar. 7, 2003.

(51) Int. Cl. C12Q 1/68; G01N 33/53

(52) U.S. Cl. 435/6; 435/7.1

(57) ABSTRACT

Polymorphic A-kinase anchor proteins (AKAPs) and nucleic acids encoding the proteins are provided herein. Methods of detecting polymorphic AKAPs and nucleic acids encoding the AKAPs, and kits for use in the detection methods are also provided. Further provided herein are methods of identifying subjects having or at risk of developing diseases or disorders, such as those related to signal transduction and/or cardiovascular disease. Methods of determining susceptibility to morbidity and/or increased or early mortality are also provided.

### RELATED APPLICATIONS

This application claims the benefit of priority to U.S. provisional application Ser. No. 60/453,215, to Andreas Braun and Stefan Kammerer entitled “ASSOCIATION OF POLYMORPHIC KINASE ANCHOR PROTEINS WITH CARDIAC PHENOTYPES AND RELATED METHODS”, filed Mar. 7, 2003, U.S. provisional application Ser. No. 60/453,208, to Andreas Braun and Stefan Kammerer entitled “ASSOCIATION OF POLYMORPHIC KINASE ANCHOR PROTEINS WITH CARDIAC PHENOTYPES AND RELATED METHODS”, filed Mar. 7, 2003, and U.S. provisional application Ser. No. 60/453,350, to Andreas Braun and Stefan Kammerer entitled “ASSOCIATION OF POLYMORPHIC KINASE ANCHOR PROTEINS WITH CARDIAC PHENOTYPES AND RELATED METHODS”, filed Mar. 7, 2003.

Where permitted, the subject matter of each of the above-noted applications is incorporated herein by reference. Also, where permitted the subject matter and disclosure of U.S. application Ser. No. 09/834,700 to Andreas Braun entitled “POLYMORPHIC KINASE ANCHOR PROTEINS AND NUCLEIC ACIDS ENCODING THE SAME”, filed Apr. 12, 2001, and U.S. application Ser. No. 10/428,254 to Andreas Braun, Charles Cantor, Stefan Kammerer, Susan Taylor, and Lora Burns, entitled “KINASE ANCHOR PROTEIN MUTEINS, PEPTIDES THEREOF, AND RELATED METHODS”, filed May 1, 2003, are incorporated by reference.

### FIELD OF THE INVENTION

Methods of identifying subjects having or at a risk of developing disorders of cellular protein phosphorylation and/or signal transduction. Methods of determining susceptibility to morbidity and/or increased or early mortality are also provided.

### BACKGROUND OF THE INVENTION

Protein phosphorylation is an important mechanism for enzyme regulation and the transduction of extracellular signals across the cell membrane in eukaryotic cells. A wide variety of cellular substrates, including enzymes, membrane receptors, ion channels and transcription factors, can be phosphorylated in response to extracellular signals that interact with cells. A key enzyme in the phosphorylation of cellular proteins in response to hormones and neurotransmitters is cyclic AMP (cAMP)-dependent protein kinase (PKA). Upon activation by cAMP, PKA thus mediates a variety of cellular responses to such extracellular signals.

An array of PKA isozymes are expressed in mammalian cells. The PKAs usually exist as inactive tetrameres containing a regulatory (R) subunit dimer and two catalytic (C) subunits. Genes encoding three C subunits (Cα, Cβ and Cγ) and four R subunits (RIα, RIβ, RIIα and RIIβ) have been identified (see Takio et al. (1982) Proc. Natl. Acad. Sci. U.S.A. 79:2544-2548; Lee et al. (1983) Proc. Natl. Acad. Sci. U.S.A. 80:3608-3612; Jahnsen et al. (1996) J. Biol. Chem. 261:12352-12361; Clegg et al. (1988) Proc. Natl. Acad. Sci. U.S.A. 85:3703-3707; and Scott (1991) Pharmacol. Ther. 50:123-145). The type I (RI) α and type II (RII) α subunits are distributed ubiquitously, whereas RIβ and RIIβ are present mainly in brain (see. e.g., Miki and Eddy (1999) J. Biol. Chem. 274:29057-29062). The type I PKA holoenzyme (RIα and RIβ) is predominantly cytoplasmic, whereas the majority of type II PKA (RIIα and RIIβ) associates with cellular structures and organelles (Scott (1991) Pharmacol. Ther. 50:123-145). Many hormones and other signals act through receptors to generate cAMP which binds to the R subunits of PKA and releases and activates the C subunits to phosphorylate proteins.

Because protein kinases and their substrates are widely distributed throughout cells, there are mechanisms in place in cells to localize protein kinase-mediated responses to different signals. One such mechanism involves subcellular targeting of PKAs through association with anchoring proteins, referred to as A-kinase anchoring proteins (AKAPs), that place PKAs in close proximity to specific organelles or cytoskeletal components and particular substrates thereby providing for more specific PKA interactions and localized responses (see, e.g., Scott et al. (1990) J. Biol. Chem. 265:21561-21566; Bregman et al. (1991) J. Biol. Chem. 266:7207-7213; and Miki and Eddy (1999) J. Biol. Chem. 274:29057-29062). Anchoring not only places the kinase close to preferred substrates, but also positions the PKA holoenzyme at sites where it can optimally respond to fluctuations in the second messenger cAMP (Mochly-Rosen (1995) Science 268:247-251; Faux and Scott (1996) Trends Biochem. Sci. 21:312-315; Hubbard and Cohen (1993) Trends Biochem. Sci. 18:172-177).

Up to 75% of type II PKA is localized to various intracellular sites through association of the regulatory subunit (RII) with AKAPs (see, e.g., Hausken et al. (1996) J. Biol. Chem. 271:29016-29022). RII subunits of PKA bind to AKAPs with nanomolar affinity (Carr et al. (1992) J. Biol. Chem. 267:13376-13382), and many AKAP-RII complexes have been isolated from cell extracts. RI subunits of PKA bind to AKAPs with only micromolar affinity (Burton et al. (1997) Proc. Natl. Acad. Sci. U.S.A. 94:11067-11072). Evidence of binding of a PKA RI subunit to an AKAP has been reported (Miki and Eddy (1998) J. Biol. Chem 273:34384-34390) in which RIα-specific and RIα/RIIα dual specificity PKA anchoring domains were identified on FSC1/AKAP82. Additional dual specific AKAPs, referred to as D-AKAP1 and D-AKAP2, which interact with the type I and type II regulatory subunits of PKA have also been reported (Huang et al. (1997) J. Biol. Chem. 272:8057-8064; Huang et al. (1997) Proc. Natl. Acad. Sci. U.S.A. 94:11184-11189).

More than 20 AKAPs have been reported in different tissues and species. Complementary DNAs (cDNAs) encoding AKAPs have been isolated from diverse species, ranging from Caenorhabditis elegans and Drosophilia to human (see, e.g., Colledge and Scott (1999) Trends Cell Biol. 9:216-221). Regions within AKAPs that mediate association with RII subunits of PKA have been identified. These regions of approximately 10-18 amino acid residues vary substantially in primary sequence, but secondary structure predictions indicate that they are likely to form an amphipathic helix with hydrophobic residues aligned along one face of the helix and charged residues along the other (Carr et al. (1991) J. Biol. Chem. 266:14188-14192; Carr et al. (1992) J. Biol. Chem. 267:13376-13382). Hydrophobic amino acids with a long aliphatic side chain, e.g., valine, leucine or isoleucine, can participate in binding to RII subunits (Glantz et al. (1993) J. Biol. Chem. 268:12796-12804).

Many AKAPs also have the ability to bind to multiple proteins, including other signaling enzymes. For example, AKAP79 binds to PKA, protein kinase C (PKC) and the protein phosphatase calcineurin (PP2B) (Coghlan et al. (1995) Science 267:108-112 and Klauck et al. (1996) Science 271:1589-1592). Therefore, the targeting of AKAP79 to neuronal postsynaptic membranes brings together enzymes with opposite catalytic activities in a single complex.

AKAPs thus serve as potential regulatory mechanisms that increase the selectivity and intensity of a cAMP-mediated response. There is a need, therefore, to identify and elucidate the structural and functional properties of AKAPs in order to gain a complete understanding of the important role these proteins play in the basic functioning of cells.

### SUMMARY

Provided herein are methods for indicating increased susceptibility of a subject to a disease or disorder. The methods include assessment of the presence or absence of an allele of the an AKAP gene and other aspects, including determining EKG features, or methods in which the AKAP gene is predictive of treatment outcome or response. Methods provided herein include steps of conducting an EKG examination; determining the EKG-PR-interval in the subject. If the EKG-PR-interval is decreased, then identity of an amino acid present in the subject at position 646 of AKAP10/D-AKAP2 (SEQ ID NO:2) or a nucleotide present at position corresponding to nucleotide 2073 of SEQ ID NO:1 is assessed. The presence of Val at position 646 of SEQ ID NO:2 or the presence of a -G- at nucleotide position 2073 of SEQ ID NO:1, indicates increased susceptibility to a disease or disorder. The disease or disorder can be selected from among a variety of diseases and discorders, including, but are not limited to, cardiovascular disorders, cardiac disease, proliferative disorders, neurological disorders, neurodegenerative disorders, obesity, diabetes and peripheral retinopathies.

Also provided are methods for indicating increased susceptibility of a subject to a disease or disorder associated with the cardiovascular system, by conducting an EKG exam; determining the EKG-PR-interval in the subject, wherein, if the EKG-PR-interval is decreased, then determining the amino acid present at position 646 of AKAP10/D-AKAP2 (SEQ ID NO:2) or the nucleotide present at position corresponding to nucleotide 2073 of SEQ ID NO:1, wherein the presence of Val at position 646 of SEQ ID NO:2 or the presence of a -G- at nucleotide position 2073 of SEQ ID NO:1, indicates increased susceptibility to a disease or disorder associated with the cardiovascular system. The EKG-PR-interval in the subject can be compared to a predetermined age-matched standard EKG-PR-interval to determine whether it is decreased.

Also provided herein are methods of assessing the susceptibility of a subject to a disease or disorder associated with the cardiovascular system, the method comprising determining the amino acid at position 646 of AKAP10/D-AKAP2 (SEQ ID NO:2) or the nucleotide present at position corresponding to nucleotide 2073 of SEQ ID NO:1, wherein the presence of Val at position 646 of SEQ ID NO:2 or the presence of a -G- at nucleotide position 2073 of SEQ ID NO:1, indicates increased susceptibility to a disease or disorder associated with the cardiovascular system.

Also provided herein are methods of diagnosing a disease or disorder associated with the cardiovascular system, comprising detecting the presence of Val at 646 of D-AKAP2 (SEQ ID NO:2) or the presence of a G at a nucleotide position corresponding to nucleotide 2073 of SEQ ID NO:1, wherein the presence of Val at position 646 of SEQ ID NO:2 or the presence of a -G- at nucleotide position 2073 of SEQ ID NO:1, indicates the presence of a disease or disorder associated with the cardiovascular system. In these methods, the disease or disorder can be from among, but not limited to, one or more of the group consisting of: atrial fibrillation, sick sinus syndrome, sudden cardiac arrest, ventricular arrythmia, ventricular fibrillation, ventricular tachycardia, Wolf-Parkinson-White (WPW) Syndrome, Lown-Ganong-Levin (LGL) Syndrome, hypertension.

Provided herein are methods for determining responsiveness of a subject to one or more, β-blocking agents, comprising detecting for the subject the presence or absence of Val at position 646 of SEQ ID NO:2 or a -G- nucleotide at a position corresponding to position 2073 of SEQ ID NO: 1, wherein the presence of a Val at position 646 of SEQ ID NO:2 or a -G- at nucleotide 2073 of SEQ ID NO:1, is indicative of an increased likelihood that a subject has a modulated response to one or more β-blocking agents compared to a subject who does not have the allelic variant. In one embodiment, the modulated response is a decreased response to one or more β-blocking agents compared to a subject who does not have the allelic variant. In another embodiment, the decreased response is a non-response to one or more β-blocking agents compared to a subject who does not have the allelic variant. In yet another embodiment, the modulated response is an increased response to one or more β-blocking agents compared to a subject who does not have the allelic variant. The β-blocker can be an antagonist of a β-adrenergic receptor. In another embodiment, the, β-blocker is an agonist of a β-adrenergic receptor.

Also provided herein are methods for determining responsiveness of a subject to one or more β-blocking agents, comprising detecting the presence or absence of Val at position 646 of SEQ ID NO:2 or a -G- nucleotide at a position corresponding to position 2073 of SEQ ID NO: 1, wherein the presence of a Val at position 646 of SEQ ID NO:2 or a -G- at nucleotide 2073 of SEQ ID NO:1, is indicative of an increased likelihood that a subject has an increased response to one or more β-blocking agents compared to a subject who does not have the allelic variant.

Also provided herein, are methods for determining responsiveness of a subject to one or more β-blocking agents, comprising detecting for the subject the presence or absence of Val at position 646 of SEQ ID NO:2 or a -G- nucleotide at a position corresponding to position 2073 of SEQ ID NO: 1, wherein the presence of a Val at position 646 of SEQ ID NO:2 or a -G- at nucleotide 2073 of SEQ ID NO:1, is indicative of an increased likelihood that a subject is non-responsive to one or more β-blocking agents compared to a subject who does not have the allelic variant.

Also provided herein are methods for determining responsiveness of a subject to one or more β-blocking agents, comprising detecting the presence or absence of Val at position 646 of SEQ ID NO:2 or a -G- nucleotide at a position corresponding to position 2073 of SEQ ID NO: 1, wherein the presence of a Val at position 646 of SEQ ID NO:2 or a -G- at nucleotide 2073 of SEQ ID NO:1, is indicative of an increased likelihood that a subject is hyper-responsive to one or more β-blocking agents compared to a subject who does not have the allelic variant.

Provided herein are methods for indicating susceptibility of a subject to acquired long Q-T syndrome, comprising detecting the presence or absence of Val at position 646 of SEQ ID NO:2 or presence or absence of a -G- nucleotide at a position corresponding to position 2073 of SEQ ID NO: 1, wherein the presence of a Val at position 646 of SEQ ID NO:2 or a -G- at nucleotide 2073 of SEQ ID NO:1, is indicative of increased susceptibility to acquired long Q-T syndrome, compared to the susceptibility of a subject who does not have the allelic variant. In accordance with this embodiment, the detecting step can be effected by a method selected from the group consisting of allele specific hybridization, primer specific extension, oligonucleotide ligation assay, restriction enzyme site analysis and single-stranded conformation polymorphism analysis. Also provided herein, the detecting step can comprise mass spectrometry. Also provided herein, detection can be effected by detecting a signal moiety selected from the group consisting of radioisotopes, enzymes, antigens, antibodies, spectrophotometric reagents, chemiluminescent reagents, fluorescent reagents and other light producing reagents.

Also provided herein, are methods for indicating susceptibility to morbidity, increased or early mortality, or morbidity and increased or early mortality of a subject; comprising conducting an EKG exam; determining the EKG-PR-interval in the subject, wherein if the EKG-PR-interval is decreased; then determining the amino acid at position 646 of AKAP10/D-AKAP2 (SEQ ID NO:2) or the nucleotide present at position corresponding to nucleotide 2073 of SEQ ID NO:1, wherein the presence of Val at position 646 of SEQ ID NO:2 or the presence of a -G- at nucleotide position 2073 of SEQ ID NO:1, indicates increased susceptibility to morbidity, increased or early mortality, or morbidity and increased or early mortality of a subject.

In one embodiment of each of the methods provided herein, the subject is heterozygous -GA- at a position corresponding to nucleotide 2073 of SEQ ID NO:1 or heterozygous Val/Ile at a position corresponding to position 646 of SEQ ID NO:2. In another embodiment of each of the methods provided herein, the subject is homozygous -GG- at a position corresponding to nucleotide 2073 of SEQ ID NO:1 or homozygous Val/Val at a position corresponding to position 646 of SEQ ID NO:2.

Another non-synonymous D-AKAP2 variation retrieved from dbSNP has been verified. The G-A transversion in exon 4 results in an Arg to His substitution at position 249 of SEQ ID NO:2 (R249H; corresponding to a G to A transversion at nucleotide 883 of SEQ ID NO:1 encoding human D-AKAP2). The Arg at residue 249 of SEQ ID NO:2 was found to be in complete linkage disequilibrium with the Ile at position 646 of SEQ ID NO:646, occurring together in every case, and therefore shows the same age effect. Accordingly, in the each of the methods provided herein, where a subject is assayed for the genotype at position 2073 of SEQ ID NO:1, the subject can also be assayed for the genotype at position 883 of SEQ ID NO:1. The genotype -G- at position 883 of SEQ ID NO:1 corresponds to genotype -A- at nucleotide 2073 of SEQ ID NO:1 and vice versa. Likewise, in the methods provided herein the genotype -A- at position 883 of SEQ ID NO:1 corresponds to genotype -G- at nucleotide 2073 of SEQ ID NO:1 and vice versa. For amino acid residues, the detection of a Ile at residue 646 of SEQ ID NO:2 corresponds to detection of an Arg at residue 249 of SEQ ID NO:2 and vice versa. Likewise, the detection of a Val at residue 646 of SEQ ID NO:2 corresponds to detection of an His at residue 249 of SEQ ID NO:2 and vice versa.

Further provided are kits for practicing the methods. The kits can include reagents for assessing genotype of an AKAP allele and also reagents and/or components for conducting an EKG and/or a β-blocking agent. For example, a kit for assessing genotype can include a primer or probe that specifically hybridizes adjacent to or at a polymorphic region spanning a position corresponding to position 2073 of SEQ ID NO 1 or 3 of an AKAP10 allele or the complement thereof and a second primer or probe that specifically hybridizes adjacent to or at a polymorphic region spanning a position corresponding to positions selected from the group consisting of position 83587 of SEQ ID NO 13 or 17, position 129600 of SEQ ID NO 14 or 17, and position 156,277 of SEQ ID NO 18 or 17 of an AKAP10 allele or the complement thereof. Primers include, but are not limited to, nucleic acids consisting essentially of the nucleotide sequence of SEQ ID NO: 8, SEQ ID NO: 15, SEQ ID NO: 19 and SEQ ID NO 20. Other genotyping components of the kit can include a first primer or probe that specifically hybridizes adjacent to or at a polymorphic region spanning a position corresponding to position 883 of SEQ ID NO 1 or 3 of an AKAP10 allele or the complement thereof and a second primer or probe that specifically hybridizes adjacent to or at a polymorphic region spanning a position corresponding to positions selected from the group consisting of position 83587 of SEQ ID NO 13 or 17, position 129600 of SEQ ID NO 14 or 17, and position 156,277 of SEQ ID NO 18 or 17 of an AKAP10 allele or the complement thereof. The kits optionally contain instructions for performing assays, interpreting results or for aiding in peforming the methods. The kits also can include at least one didieoxynucleotide such as ddA, ddC, ddG.

### BRIEF DESCRIPTION OF THE FIGURES

FIG. 1 shows the results of an analysis of covariance that was conducted to test the effect of the genotypes on PR mean levels. Age was included as a covariate in the model, which was significantly associated with PR mean up to a third order polynomial. The relationship between age and PR mean was genotype-dependent, and therefore interaction terms between genotype and age were included. The predicted values from the resulting model are shown in FIG. 1 for each genotype.

### DETAILED DESCRIPTION

A. Definitions

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, the phrase “EKG examination” or “ECG examination” refers to the well-known electrocardiogram examination that generates an electrical recording of the heart and is conducted on human subjects to investigate heart function and heart disease.

As used herein, the phrase “PR-interval” or “EKG-PR-interval”, in the context of an electrocardiogram (EKG or ECG) analysis, is the time (typically expressed herein in units of milliseconds) elapsed between the beginning of the P wave to the beginning of the next QRS complex. It corresponds to the time lag from the onset of atrial depolarization to the onset of ventricular depolarization. This time lag allows atrial systole to occur, filling the ventricles before ventricular systole. Most of the delay occurs in the AV node. The PR interval is longer with high vagal tone. A prolonged PR interval corresponds to impaired AV conduction. The normal range of PR-intervals from about 120 to 200 milliseconds. It is well-known that each square on a EKG readout (graph) corresponds to 40 milliseconds.

As used herein the phrase “predetermined standard” refers to an average of a multiplicity of EKG-PR-intervals that can be empirically determined from a specifically chosen group of individuals. The group of individuals can be selected irrespective of disease status, e.g., from a healthy patient database. In another embodiment, the standard can be obtained from a group of control age-matched subjects that do not have a particular disease, such as heart disease. In another embodiment, the predetermined standard can be obtained from a known age-matched control that is homozygous -AA- at a position corresponding to nucleotide 2073 of SEQ ID NO:1 or homozygous Ile/Ile at a position corresponding to position 646 of SEQ ID NO:2.

As used herein “age-matched standard EKG-PR-interval” refers to the average PR-interval (also referred to herein as “PRmean”) for a multiplicity of subjects of the same age. In addition, the average PR-interval can be obtained from controls having the same genotype, such as -AA- homozygotes and/or -GA- heterozygotes at a position corresponding to nucleotide 2073 of SEQ ID NO:1. Accordingly, the EKG-PR-interval can be stratified by age and/or genotype. For example, the EKG-PR-interval of the subject being examined can be compared to PRmean of either: a group of control subjects of the same age having the -AA- homozygous genotype at a position corresponding to nucleotide 2073 of SEQ ID NO:1; a group of control subjects of the same age having the -GA- heterozygous genotype at a position corresponding to nucleotide 2073 of SEQ ID NO:1; both groups of control subjects of the same age having either the -AA- homozygous or the -GA- homozygous genotype at a position corresponding to nucleotide 2073 of SEQ ID NO:1. In another embodiment, the EKG-PR-interval of the subject being examined can be compared to the PRmean of a group of control subjects of the same age having any genotype at a position corresponding to nucleotide 2073 of SEQ ID NO:1.

As used herein, the phrase “disease or disorders” is meant to encompass all genetic or physiological irregularities that can be attributed to a particular body organ or physiological or cellular system. As used herein, the phrase “disorder or disease associated with a decreased EKG-PR-interval” or grammatical variations thereof, refers to any disease or disorder that exhibits a decreased PR-interval as one of its characteristics. Exemplary disorders and/or diseases contemplated herein as associated with decreased EKG-PR-intervals include, but are not limited to, those involving alterations in cellular protein phosphorylation and/or signal transduction. Among such disorders and diseases are: neurodegeneratives diseases, such as Alzheimer's Disease, cardiovascular disorders, cardiac disorders, particularly disorders associated with altered left ventricular function, cardiomyopathies, proliferative disorders, bipolar disorder and other neurological disorders, obesity, diabetes and certain peripheral retinopathies, such as retinitis pigmentosa. As used herein, cardiovascular disorders or cardiac disease collectively encompass all cardiovascular abnormalities, such as, but not limited to congenital heart disease, cardiac arrhythmia, brachycardia, atrial fibrillation, sick sinus syndrome, sudden cardiac arrest, ventricular arrythmia, ventricular fibrillation, ventricular tachycardia, Wolf-Parkinson-White (WPW) Syndrome, Lown-Ganong-Levin (LGL) Syndrome, hypertension, familial cardiac myxomas and Carney complex.

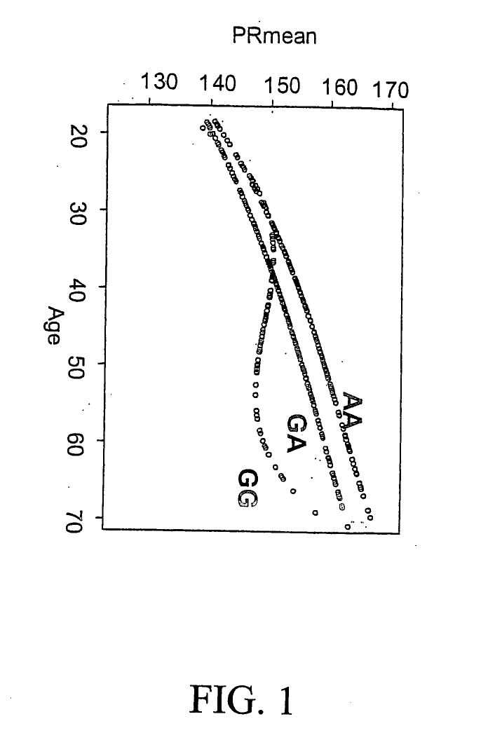

As used herein, a “decreased” EKG-PR-interval or grammatical variations thereof, refers to a PR-interval that is lower than the average PR-interval (e.g., PRmean) for subjects of the same age group. As set forth herein (e.g., FIG. 1) the average PR-interval (e.g., PRmean) increases with age for subjects having either a homozygous -AA- or heterozygous -GA- genotype at a position corresponding to nucleotide 2073 of SEQ ID NO:1 encoding the AKAP10/D-AKAP2 protein. For subjects (e.g., humans) having the homozygous -GG- genotype at a position corresponding to nucleotide 2073 of SEQ ID NO:1, it has been found herein that the average PR-interval is decreased relative to subjects of the same age that have either a homozygous -AA- or heterozygous -GA- genotype. The decreased EKG-PR-interval is evident in subjects from the age of about 40 up to about 70 years of age.

In addition, although the average PR-interval for the -GA- heterozygotes increases with age as with the -AA- homozygotes, the average PR-interval (e.g., PRmean) is always lower for the -GA- heteroaygotes than for the -AA- homozygotes (see FIG. 1). Accordingly, in one embodiment, the EKG-PR-interval of the subject being examined is compared to the PRmean (e.g., average PR-interval) of age-matched -AA- homozygotes. This embodiment serves as a preliminary screen for subjects that have either 1 or 2 copies of the 1646V variant (e.g., screen for potential -GA- heterozygotes or -AA- homozygotes at a nucleotide position corresponding to nucleotide 2073 of SEQ ID NO:1). In another embodiment, the EKG-PR-interval of the subject being examined is compared to the PRmean (e.g., average PR-interval) of age-matched -GA- heterozygotes. This embodiment serves as preliminary screen for subjects that have 2 copies of the I646V variant, e.g., subjects that are -GG- homozygotes.

In the methods provided herein, if the EKG-PR-interval is not decreased relative to the age-matched average PR-interval (PRmean) for -AA- homozygotes and/or -GA- heterozygotes at a position corresponding to nucleotide 2073 of SEQ ID NO:1, then there is no need to determine the genotype of the subject.

As used herein, a Q-T interval is the time from electrocardiogram Q wave to the end of the T wave corresponding to electrical systole. This interval represents the time required for depolarization and repolarization to occur. In long QT syndrome, the duration of repolarization is longer than normal. Thus, the QT-interval is prolonged. An interval above 440 milliseconds (msec) is considered prolonged. An interval at or above 480 milliseconds in females or 470 milliseconds in males typically is sufficient to diagnose a subject as having long QT syndrome.

As used herein, sequencing refers to the process of determining a nucleotide sequence and can be performed using any method known to those of skill in the art. For example, if a polymorphism is identified or known, and it is desired to assess its frequency or presence in nucleic acid samples taken from the subjects that comprise the database, the region of interest from the samples can be isolated, such as by PCR or restriction fragments, hybridization or other suitable method known to those of skill in the art, and sequenced. For purposes herein, sequencing analysis can be carried out using mass spectrometry (see, e.g., U.S. Pat. Nos. 5,547,835, 5,622,824, 5,851,765, and 5,928,906). Nucleic acids can also be sequenced by hybridization (see, e.g., U.S. Pat. Nos. 5,503,980, 5,631,134, 5,795,714) and including analysis by mass spectrometry (see, U.S. application Ser. Nos. 08/419,994 and 09/395,409). Alternatively, sequencing can be performed using other known methods, such as set forth in U.S. Pat. Nos. 5,525,464; 5,695,940; 5,834,189; 5,869,242; 5,876,934; 5,908,755; 5,912,118; 5,952,174; 5,976,802; 5,981,186; 5,998,143; 6,004,744; 6,017,702; 6,018,041; 6,025,136; 6,046,005; 6,087,095; 6,117,634, 6,013,431, WO 98/30883; WO 98/56954; WO 99/09218; WO/00/58519, and the others.

As used herein, “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides in length.

As used herein, “polymorphic gene” refers to a gene having at least one polymorphic region.

As used herein, “allele”, which is used interchangeably herein with “allelic variant” refers to alternative forms of a gene or portions thereof. Alleles occupy the same locus or position on homologous chromosomes. When a subject has two identical alleles of a gene, the subject is the to be homozygous for the gene or allele. When a subject has two different alleles of a gene, the subject is the to be heterozygous for the gene. Alleles of a specific gene can differ from each other in a single nucleotide, or several nucleotides, and can include substitutions, deletions, and insertions of nucleotides. An allele of a gene can also be a form of a gene containing a mutation.

As used herein, “predominant allele” refers to an allele that is represented in the greatest frequency for a given population. The allele or alleles that are present in lesser frequency are referred to as allelic variants.

As used herein, “associated” refers to coincidence with the development or manifestation of a disease, condition or phenotype. Association can be due to, but is not limited to, genes responsible for housekeeping functions whose alteration can provide the foundation for a variety of diseases and conditions, those that are part of a pathway that is involved in a specific disease, condition or phenotype and those that indirectly contribute to the manifestation of a disease, condition or phenotype.

As used herein, the term “subject” refers to mammals and in particular human beings.

As used herein, the term “gene” or “recombinant gene” refers to a nucleic acid molecule comprising an open reading frame and including at least one exon and (optionally) an intron sequence. A gene can be either RNA or DNA. Genes can include regions preceding and following the coding region (leader and trailer).

As used herein, “intron” refers to a DNA sequence present in a given gene which is spliced out during mRNA maturation.

As used herein, “nucleotide sequence complementary to the nucleotide sequence set forth in SEQ ID NO: x” refers to the nucleotide sequence of the complementary strand of a nucleic acid strand having SEQ ID NO: x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID NO: x refers to the complementary strand of the strand having SEQ ID NO: x or to any nucleic acid having the nucleotide sequence of the complementary strand of SEQ ID NO: x. When referring to a single stranded nucleic acid having the nucleotide sequence SEQ ID NO: x, the complement of this nucleic acid is a nucleic acid having a nucleotide sequence which is complementary to that of SEQ ID NO: x.

As used herein, the term “coding sequence” refers to that portion of a gene that encodes an amino acid sequence of a protein.

As used herein, the term “sense strand” refers to that strand of a double-stranded nucleic acid molecule that has the sequence of the mRNA that encodes the amino acid sequence encoded by the double-stranded nucleic acid molecule.

As used herein, the term “antisense strand” refers to that strand of a double-stranded nucleic acid molecule that is the complement of the sequence of the mRNA that encodes the amino acid sequence encoded by the double-stranded nucleic acid molecule.

As used herein, the amino acids, which occur in the various amino acid sequences appearing herein, are identified according to their well-known, three-letter or one-letter abbreviations. The nucleotides, which occur in the various DNA fragments, are designated with the standard single-letter designations used routinely in the art (see, Table 1).

As used herein, amino acid residue refers to an amino acid formed upon chemical digestion (hydrolysis) of a polypeptide at its peptide linkages. The amino acid residues described herein are typically in the “L” isomeric form. Residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§1.821-1.822, abbreviations for amino acid residues are shown in the following Table:

TABLE 1
Table of Correspondence

SYMBOL

| | | |
|---|---|---|
| 1-Letter | 3-Letter | AMINO ACID |

| | | |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224).

Such substitutions are typically made in accordance with those set forth in TABLE 2 as follows:

TABLE 2

| | |
|---|---|
| Original residue | Conservative substitution |

| | |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions are also permissible and can be determined empirically or in accord with known conservative substitutions.

As used herein, a DNA or nucleic acid homolog refers to a nucleic acid that includes a preselected conserved nucleotide sequence, such as a sequence encoding a therapeutic polypeptide. By the term “substantially homologous” is meant having at least 80%, typically at least 90%, or at least 95% homology therewith or a less percentage of homology or identity and conserved biological activity or function.

The terms “homology” and “identity” are often used interchangeably. In this regard, percent homology or identity can be determined, for example, by comparing sequence information using a GAP computer program. The GAP program uses the alignment method of Needleman and Wunsch (J. Mol. Biol. 48:443 (1970), as revised by Smith and Waterman (Adv. Appl. Math. 2:482 (1981). Briefly, the GAP program defines similarity as the number of aligned symbols (i.e., nucleotides or amino acids) which are similar, divided by the total number of symbols in the shorter of the two sequences. The default parameters for the GAP program can include: (1) a unary comparison matrix (containing a value of 1 for identities and 0 for non-identities) and the weighted comparison matrix of Gribskov and Burgess, Nucl. Acids Res. 14:6745 (1986), as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Whether any two nucleic acid molecules have nucleotide sequences that are at least 80%, 85%, 90%, 95%, 96%, 97%, 98% or 99% “identical” can be determined using known computer algorithms such as the “FAST A” program, using for example, the default parameters as in Pearson and Lipman, Proc. Natl. Acad. Sci. USA 85:2444 (1988). Alternatively the BLAST function of the National Center for Biotechnology Information database can be used to determine identity

In general, sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)). Methods commonly employed to determine identity or similarity between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and similarity are codified in computer programs. Typical computer program methods to determine identity and similarity between two sequences include, but are not limited to, GCG program package (Devereux, J., et al., Nucleic Acids Research 12(I):387 (1984)), BLASTP, BLASTN, FASTA (Atschul, S. F., et al., J Molec Biol 215:403 (1990)).

Therefore, as used herein, the term “identity” represents a comparison between a test and a reference polypeptide or polynucleotide. For example, a test polypeptide can be defined as any polypeptide that is 90% or more identical to a reference polypeptide.

As used herein, the term at least “90% identical to” refers to percent identities from 90 to 99.99 relative to the reference polypeptides. Identity at a level of 90% or more is indicative of the fact that, assuming for exemplification purposes a test and reference polypeptide length of 100 amino acids are compared. No more than 10% (i.e., 10 out of 100) amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g. 10/100 amino acid difference (approximately 90% identity). Differences are defined as nucleic acid or amino acid substitutions, or deletions.

As used herein, stringency conditions refer to the washing conditions for removing the non-specific probes and conditions that are equivalent to either high, medium, or low stringency as described below:

| | |
|---|---|
| 1) high stringency: | 0.1 × SSPE, 0.1% SDS, 65° C. |
| 2) medium stringency: | 0.2 × SSPE, 0.1% SDS, 50° C. |
| 3) low stringency: | 1.0 × SSPE, 0.1% SDS, 50° C. |

It is understood that equivalent stringencies can be achieved using alternative buffers, salts and temperatures.

As used herein, “heterologous DNA” is DNA that encodes RNA and proteins that are not normally produced in vivo by the cell in which it is expressed or that mediates or encodes mediators that alter expression of endogenous DNA by affecting transcription, translation, or other regulatable biochemical processes or is not present in the exact orientation or position as the counterpart DNA in a wildtype cell. Heterologous DNA can also be referred to as foreign DNA. Any DNA that one of skill in the art would recognize or consider as heterologous or foreign to the cell in which is expressed is herein encompassed by heterologous DNA. Examples of heterologous DNA include, but are not limited to, DNA that encodes traceable marker proteins, such as a protein that confers drug resistance, DNA that encodes therapeutically effective substances, such as anti-cancer agents, enzymes and hormones, and DNA that encodes other types of proteins, such as antibodies. Antibodies that are encoded by heterologous DNA can be secreted or expressed on the surface of the cell in which the heterologous DNA has been introduced.

As used herein, isolated with reference to a nucleic acid molecule or polypeptide or other biomolecule means that the nucleic acid or polypeptide has separated from the genetic environment from which the polypeptide or nucleic acid were obtained. It can also mean altered from the natural state. For example, a polynucleotide or a polypeptide naturally present in a living animal is not “isolated,” but the same polynucleotide or polypeptide separated from the coexisting materials of its natural state is “isolated”, as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been purified, partially or substantially, from a recombinant host cell or from a native source. For example, a recombinantly produced version of a compound can be substantially purified by the one-step method described in Smith and Johnson, Gene 67:31-40 (1988). The terms isolated and purified are sometimes used interchangeably.

Thus, by “isolated” is meant that the nucleic acid is free of the coding sequences of those genes that, in the naturally-occurring genome of the organism (if any) immediately flank the gene encoding the nucleic acid of interest. Isolated DNA can be single-stranded or double-stranded, and can be genomic DNA, cDNA, recombinant hybrid DNA, or synthetic DNA. It can be identical to a native DNA sequence, or can differ from such sequence by the deletion, addition, or substitution of one or more nucleotides.

Isolated or purified as it refers to preparations made from biological cells or hosts means any cell extract containing the indicated DNA or protein including a crude extract of the DNA or protein of interest. For example, in the case of a protein, a purified preparation can be obtained following an individual technique or a series of preparative or biochemical techniques and the DNA or protein of interest can be present at various degrees of purity in these preparations. The procedures can include for example, but are not limited to, ammonium sulfate fractionation, gel filtration, ion exchange change chromatography, affinity chromatography, density gradient centrifugation and electrophoresis.

A preparation of DNA or protein that is “substantially pure” or “isolated” should be understood to mean a preparation free from naturally occurring materials with which such DNA or protein is normally associated in nature. “Essentially pure” should be understood to mean a “highly” purified preparation that contains at least 95% of the DNA or protein of interest.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

As used herein, receptor refers to a biologically active molecule that specifically binds to (or with) other molecules. The term “receptor protein” can be used to more specifically indicate the proteinaceous nature of a specific receptor.

As used herein, recombinant refers to any progeny formed as the result of genetic engineering.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences can be cis acting or can be responsive to trans acting factors. Promoters, depending upon the nature of the regulation, can be constitutive or regulated.

As used herein, the phrase “operatively linked” generally means the sequences or segments have been covalently joined into one piece of DNA, whether in single or double stranded form, whereby control or regulatory sequences on one segment control or permit expression or replication or other such control of other segments. The two segments are not necessarily contiguous. For gene expression a DNA sequence and a regulatory sequence(s) are connected in such a way to control or permit gene expression when the appropriate molecular, e.g., transcriptional activator proteins, are bound to the regulatory sequence(s).

As used herein, production by recombinant means by using recombinant DNA methods means the use of the well known methods of molecular biology for expressing proteins encoded by cloned DNA, including cloning expression of genes and methods, such as gene shuffling and phage display with screening for desired specificities.

As used herein, the term “conjugated” refers stable attachment, such ionic or covalent attachment.

As used herein, a composition refers to any mixture of two or more products or compounds. It can be a solution, a suspension, liquid, powder, a paste, aqueous, non-aqueous or any combination thereof.

As used herein, a combination refers to any association between two or more items.

As used herein, substantially identical to a product means sufficiently similar so that the property of interest is sufficiently unchanged so that the substantially identical product can be used in place of the product.

As used herein, the term “vector” refers to a nucleic acid molecule capable of transporting another nucleic acid to which it has been linked. One typical vector is an episome, i.e., a nucleic acid capable of extra-chromosomal replication. Typical vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. “Plasmid” and “vector” are used interchangeably as the plasmid is the most commonly used form of vector. Other such other forms of expression vectors that serve equivalent functions and that become known in the art subsequently hereto.

As used herein, “indicating” or “determining” means that the presence or absence of an allelic variant can be one of many factors that are considered when a subject's predisposition to a disease or disorder is evaluated. Thus a predisposition to a disease or disorder is not necessarily conclusively determined by only ascertaining the presence or absence of one or more allelic variants, but the presence of one of more of such variants is among a number of factors considered.

As used herein, “predisposition to develop a disease or disorder” means that a subject having a particular genotype and/or haplotype has a higher likelihood than one not having such a genotype and/or haplotype for developing a particular disease or disorder.

As used herein, “morbidity” refers to conditions, such as diseases or disorders, that compromise the health and well-being of an organism, such as an animal. Morbidity susceptibility or morbidity-associated genes are genes that, when altered, for example, by a variation in nucleotide sequence, facilitate the expression of a specific disease clinical phenotype. Thus, morbidity susceptibility genes have the potential, upon alteration, of increasing the likelihood or general risk that an organism will develop a specific disease.

As used herein, “mortality” refers to the statistical likelihood that an organism, particularly an animal, will not survive a full predicted lifespan. Hence, a trait or a marker, such as a polymorphism, associated with increased mortality is observed at a lower frequency in older than younger segments of a population.

As used herein, “transgenic animal” refers to any animal, typically a non-human animal, e.g. a mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule can be integrated within a chromosome, or it can be extrachromosomally replicating DNA. In the typical transgenic animals described herein, the transgene causes cells to express a recombinant form of a protein. Transgenic animals in which the recombinant gene is silent are also contemplated, as for example, using the FLP or CRE recombinase dependent constructs. Moreover, “transgenic animal” also includes those recombinant animals in which gene disruption of one or more genes is caused by human intervention, including recombination and antisense techniques.

As used herein, “target nucleic acid” refers to a nucleic acid molecule which contains all or a portion of a polymorphic region of a gene of interest.

As used herein, “signal moiety” refers to any moiety that allows for the detection of a nucleic acid molecule. Included are moieties covalently attached to nucleic acids and those that are not.

As used herein, “molecule that modulates or effects the biological activity of an AKAP10 protein” refers to any drug, small molecule, nucleic acid (sense and antisense), ribozyme, protein, peptide, lipid, carbohydrate etc. or combination thereof, that directly or indirectly changes, alters, abolishes, increases or decreases a biological activity attributed to AKAP10 protein.

As used herein, “biological activity of an AKAP10 protein” refers to, but is not limited to, binding of AKAP10 to protein kinase A or its subunits, localization of AKAP10 protein to a subcellular site, e.g., the mitochondria, localization of protein kinase A to the mitochondria and binding of AKAP10 protein to other proteins including other signaling enzymes.

As used herein, “combining” refers to contacting the biologically active agent with a cell or animal such that the agent is introduced into the cell or animal. For a cell any method that results in an agent traversing the plasma membrane is useful. For an animal any of the standard routes of administration of an agent, e.g. oral, rectal, transmucosal, intestinal, intravenous, intraperitoneal, intraventricular, subcutaneous, intramuscular, etc., can be used.

As used herein, a composition refers to any mixture. It can be a solution, a suspension, liquid, powder, a paste, aqueous, non-aqueous or any combination thereof.

As used herein, a combination refers to any association between two or among more items.

As used herein, “kit” refers to a package that contains a combination, such as one or more primers or probes used to amplify or detect polymorphic regions of AKAP10 genes, optionally including instructions and/or reagents for their use.

As used herein, “solid support” refers to a support substrate or matrix, such as silica, polymeric materials or glass. At least one surface of the support can be partially planar. Regions of the support can be physically separated, for example with trenches, grooves, well or the like. Some examples of solid supports include slides and beads. Supports are of such composition so as to allow for the immobilization or attachment of nucleic acids and other molecules such that these molecules retain their binding ability.

As used herein, “array” refers to a collection of elements, such as nucleic acids, containing three or more members. An addressable array is one in which the members of the array are identifiable, typically by position on a solid support. Hence, in general the members of the array will be immobilized to discrete identifiable loci on the surface of a solid phase.

As used herein, “specifically hybridizes” refers to hybridization of a probe or primer only to a target sequence preferentially to a non-target sequence. Those of skill in the art are familiar with parameters that affect hybridization; such as temperature, probe or primer length and composition, buffer composition and salt concentration and can readily adjust these parameters to achieve specific hybridization of a nucleic acid to a target sequence.

As used herein “nucleic acid” refers to polynucleotides such as deoxyribonucleic acid (DNA) and ribonucleic acid (RNA). The term should also be understood to include, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine.

As used herein, “mass spectrometry” encompasses any suitable mass spectrometric format known to those of skill in the art. Such formats include, but are not limited to, Matrix-Assisted Laser Desorption/Ionization, Time-of-Flight (MALDI-TOF), Electrospray (ES), IR-MALDI (see, e.g., published International PCT Application No. WO 99/57318 and U.S. Pat. No. 5,118,937) Ion Cyclotron Resonance (ICR), Fourier Transform and combinations thereof. MALDI, particular UV and IR, are among the typical formats.

As used herein, “at a position corresponding to” refers to a position of interest (i.e., base number or residue number) in a nucleic acid molecule or protein relative to the position in another reference nucleic acid molecule or protein. Corresponding positions can be determined by comparing and aligning sequences to maximize the number of matching nucleotides or residues, for example, such that identity between the sequences is greater than 95%, greater than 96%, greater than 97%, greater than 98%, or greater than 99%. The position of interest is then given the number assigned in the reference nucleic acid molecule. For example, it is shown herein that a particular polymorphism in AKAP10 occurs at nucleotide 2073 of SEQ ID No. 1. To identify the corresponding nucleotide in another allele or isolate, the sequences are aligned and then the position that lines up with 2073 is identified. Since various alleles can be of different length, the position designate 2073 can not be nucleotide 2073, but instead is at a position that “corresponds” to the position in the reference sequence.

As used herein, “primer” and “probe” refer to a nucleic acid molecule including DNA, RNA and analogs thereof, including protein nucleic acids (PNA), and mixtures thereof. Such molecules are typically of a length such that they are statistically unique (i.e., occur only once) in the genome of interest. Generally, for a probe or primer to be unique in the human genome, it contains at least 14, 16 or contiguous nucleotides of a sequence complementary to or identical to a gene of interest. Probes and primers can be 10, 20, 30, 50, 100 or more nucleic acids long.

As used herein, “antisense nucleic acid molecule” refers to a molecule encoding a sequence complementary to at least a portion of an RNA molecule. The sequence is sufficiently complementary to be able to hybridize with the RNA, typically under moderate or high stringency conditions to form a stable duplex. The ability to hybridize depends on the degree of complementarity and the length of the antisense nucleic acid. Generally, the longer the hybridizing nucleic acid, the more base mismatches with an RNA it can contain and still form a stable duplex. One skilled in the art can ascertain a tolerable degree of mismatch by use of standard procedures to determine the melting point of the hybridized complex.

As used herein, a “variant protein” refers to a protein encoded by an allelic variant of a AKAP10 gene which results in a change of an amino acid residue at a particular position relative to that position in the protein encoded by the predominant allele.

As used herein, “signal transduction” refers to the propagation of a signal. In general, an extracellular signal is transmitted through the cell membrane to become an intracellular signal. This signal can then stimulate a cellular response. The term also encompasses signals that are propagated entirely within a cell. The polypeptide molecules involved in signal transduction processes are typically receptor and non-receptor protein kinases, receptor and non-receptor protein phosphatases, nucleotide exchange factors and transcription factors. One of the key biochemical mechanisms involved in signal transduction is protein phosphorylation. AKAP10 proteins are involved in signal transduction as they bind to protein kinase A (PKA) and are though to anchor the kinase at a location, e.g., the mitochondria, where PKA acts to phosphorylate a specific substrate. Thus, an alteration in AKAP10 binding to PKA, localization to the mitochondria, or phosphorylation by PKA, among other steps will result in an alteration in signal transduction. Assays including those that determine phosphorylation by PKA, association of PKA and AKAP10 and localization of AKAP10 can be used to monitor the state of signal transduction.

As used herein, “adjacent” refers to a position 5′ to the site of a single nucleotide polymorphism (SNP) such that there could be unpaired nucleotides between that position and the site of the SNP.

As used herein, “immediately adjacent” refers to a position 5′ to the site of a single nucleotide polymorphism (SNP) such that there are no unpaired nucleotides between that position and the site of the SNP.

As used herein, “binding to PKA”, refers to the interaction of the PKA binding domain of an AKAP10 protein and the regulatory subunits RI and/or RII of the protein kinase A holoenzyme.

B. Methods Employing Polymorphic AKAPs

Methods herein include a step a identifying the prescence of a particular allele of an A-kinase anchoring protein (AKAP) genes. Thus, polymorphic sequences encoding an A-kinase anchoring protein (AKAP) genes and polymorphic AKAP proteins encoded by polymorphic AKAP gene sequences are used in methods provided herein. These polymorphic sequences are based on differences in AKAP genes within and among different organisms, including humans.

Polymorphisms of the genome can lead to altered gene function, protein function or mRNA instability. AKAPs provide a mechanism for regulating ubiquitous cAMP-dependent kinase (PKA) activity by tethering PKA to specific subcellular locations thereby segregating it with particular components in a given signaling pathway and contributing to specificity in cellular responses to extracellular signals. AKAPs thus play a fundamental role in the basic functioning of cells, the response of cells to their environment and ultimately in the coordination of vital systems within an organism. Therefore, polymorphisms in AKAP gene sequences can affect the proper functioning of cells and systems within organisms and could be directly linked with certain disorders or could predispose an organism to a variety of diseases and disorders, especially those involving alterations in cellular protein phosphorylation and/or signal transduction. Among such disorders and diseases include, but are not limited to, neurodegeneratives diseases, such as Alzheimer's Disease, cardiovascular disorders, cardiac disorders, particularly disorders associated with altered left ventricular function, cardiomyopathies, proliferative disorders, bipolar disorder and other neurological disorders, obesity, diabetes and certain peripheral retinopathies, such as retinitis pigmentosa. AKAP gene polymorphisms, such as those described herein, provides for the identification and development of diagnostic and prognostic methods, also provided herein, and the development of drug therapies and treatment regimens. Furthermore, polymorphisms of AKAP genes aid in the study of AKAP protein structure and function, which also contributes to the development of diagnostic methods and therapies.

1. AKAP10

The AKAP10 protein is primarily located in mitochondria. The sequence of a human AKAP10 cDNA (also referred to as D-AKAP2) is available in the GenBank database, at accession numbers AF037439 and NM 007202, and is provided in SEQ. ID. NO:1. The AKAP10 gene is located on chromosome 17.

The sequence of a mouse D-AKAP2 cDNA is also available in the GenBank database (see accession number AF021833). The mouse D-AKAP2 protein contains an RGS domain near the amino terminus that is characteristic of proteins that interact with Gα subunits and possess GTPase activating protein-like activity (Huang et al. (1997) Proc. Natl. Acad. Sci. U.S.A. 94:11184-11189). The human AKAP10 protein also has sequences homologous to RGS domains. The carboxy-terminal 40 residues of the mouse D-AKAP2 protein are responsible for the interaction with the regulatory subunits of PKA. This sequence is fairly well conserved between the mouse D-AKAP2 and human AKAP10 proteins.

2. Polymorphisms of the Human AKAP10 Gene and Polymorphic AKAP10 Proteins

Polymorphisms of AKAP genes that alter gene expression, regulation, protein structure and/or protein function are more likely to have a significant effect on the regulation of enzyme (particularly PKA) activity, cellular transduction of signals and responses thereto and on the basic functioning of cells than polymorphisms that do not alter gene and/or protein function. Included in the polymorphic AKAPs provided herein are human AKAP10 proteins containing differing amino acid residues at position number 646 of SEQ. ID. No. 2.

Amino acid 646 of the human AKAP10 protein (SEQ. ID. NO: 2) is located in the carboxy-terminal region of the protein within a segment that participates in the binding of R-subunits of PKAs. This segment includes the carboxy-terminal 40 amino acids.

The amino acid residue reported for position 646 of the human AKAP10 protein is an isoleucine. Polymorphic human AKAP10 proteins provided herein have the amino acid sequence set forth in SEQ. ID. NO: 2 but contain residues other than isoleucine at amino acid position 646 of the protein. In particular embodiments of the polymorphic human AKAP10 proteins provided herein, the amino acid at position 646 of SEQ. ID. NO: 2 is a valine (as set forth in SEQ. ID. NO: 4), leucine or phenylalanine residue.

a. An A to G Transition at Nucleotide 2073 of the Human AKAP10 Coding Sequence

As described herein, an allelic variant of the human AKAP10 gene is at the polymorphic site at position 2073 of the coding sequence (see SEQ. ID. NO: 3) and encodes a valine at position 646 of the AKAP10 protein. This allelic variant has been found to vary in frequency in DNA samples from younger and older segments of a healthy population. This allele has the A at position 2073 of the AKAP10 gene coding sequence of SEQ. ID. NO: 1 changed to a G, giving rise to the sequence set forth in SEQ. ID. NO: 3. Consequently, the codon for amino acid 646 changes from ATT, coding for isoleucine, to GTT, coding for valine.

b. An A to C Transversion at Nucleotide 2073 of the Human AKAP10 Coding Sequence

In another human AKAP10 allelic variant, the nucleotide at position 2073 of the coding sequence in SEQ. ID. NO: 1 is changed from an A to a C. Thus, changing the codon for amino acid 646 from ATT, coding for isoleucine, to CTT, coding for leucine.

c. An A to T Transversion at Nucleotide 2073 of the Human AKAP10 Coding Sequence

In another human AKAP10 allelic variant, the nucleotide at position 2073 of the coding sequence in SEQ. ID. NO: 1 is changed from an A to a T. Thus, the codon for amino acid 646 changes from ATT, coding for isoleucine, to TTT, coding for phenylalanine.

d. Other AKAP10 Polymorphisms

TABLE 3

| Name | GenBank Accession No. | SNP | Location |
|---|---|---|---|
| 10-1 | AC005730 | T/C | 156277 |
| 10-7 | AC005730 | G/A | 129600 |

For AKAP10-1 additional variants are represented by the presence of A or G at nucleotide position 156277 of SEQ ID NO: 17.

For AKAP10-7 additional variants are represented by the presence of C or T at nucleotide position 129600 of SEQ ID NO: 17.

C. Association of AKAP10-5 I646V Variant with Cardiac Traits

The SNPs found to be associated with age were analyzed for association with disease-related quantitative traits in a twin collection. To identify traits correlated with the observed age association of the I646V SNP, a cohort of 417 fasting Caucasian twin pairs with extensive coverage for a variety of disease-related traits was utilized. The analysis was conducted using a quantitative transmission-disequilibrium test (QTDT) as described by Abecasis et al., (Am. J. Hum. Genet., 66:279-292, 2000) to take advantage of the twin-based sample and to control for admixture and other non-genetic sources of variation. Of the 97 traits analyzed, only the PR-interval from electrocardiogram (EKG or ECG) analysis was statistically significant at a nominal level of 0.05. The PR interval from EKG analysis is referred to herein is “EKG-PR-interval”. The estimate from the QTDT model of the average effect of the G allele (Val at position 646 of SEQ ID NO:2) was to decrease the PR interval 6.3 units (P=0.007). The genotype mean values in the subset of 207 informative twin pairs were 157±23.4, 152±26.9, and 146±25.4 (mean ± standard deviation) for genotypes AA, GA, and GG, respectively.

An analysis of covariance was also conducted to test the effect of the genotypes on PR mean levels. Age was included as a covariate in the model, which was significantly associated with PR mean up to a third order polynomial as set forth in FIG. 1. The relationship between age and PR mean was genotype-dependent, and therefore interaction terms between genotype and age were included. The predicted values from the resulting model are shown in FIG. 1 for each genotype. From about the age of 30 or just beyond and up to about the age of 70, it has been found that human subjects having the -GG- genotype, on average have a lower PRmean than human subjects having an -AA- homozygous genotype at a nucleotide corresponding to position 2073 of SEQ ID NO:1. From about the age of 40, it has also been found that human subjects having the -GG- genotype, on average have a lower PRmean than human subjects having either a -GA- heterozygous or -AA- homozygous genotype at a nucleotide corresponding to position 2073 of SEQ ID NO:1. Accordingly, on average a human subject about 30 years or older having a lower PRmean than an age-matched control group of -AA- homozygous at position 2073 of SEQ ID NO:1 has a higher likelihood of having either a -GA- heterozygous or a -GG- homozygous genotype at that position.

Thus, methods of comparing a subjects PR-interval to the PRmean of an age-matched control group of either one or both of an -AA- homozygous or -GA- heterozygous genotype at a nucleotide corresponding to position 2073 of SEQ ID NO:1 can identify subjects that have a higher likelihood of possessing a -GG- homozygous genotype at that position. Once a particular subject is identified as having a lower EKG-PR-interval than the PRmean from an age-matched control group, then that subject's particular genotype can be determined using the methods provided herein at a position corresponding to nucleotide 2073 of SEQ ID NO:1. As set forth herein, those subjects having the -GG- genotype have an increase susceptibility to a disease or disorder, such as a cardiovascular disease or disorder.

As also evident from FIG. 1, on average a human subject at any age having a lower PRmean than an age-matched control group of an -AA- homozygous at a nucleotide corresponding to position 2073 of SEQ ID NO:1 has a higher likelihood of having a -GA- heterozygous genotype than a -AA- homozygous genotype. Thus, for embodiments where it would be useful to identify subjects that can contain at least one “G” allele at a nucleotide corresponding to position 2073 of SEQ ID NO:1, methods of comparing a subjects PR-interval to the PRmean of an age-matched control group of an -AA- homozygous genotype at a nucleotide corresponding to position 2073 of SEQ ID NO:1 can identify subjects that have a higher likelihood of possessing at least one -G- allele at that position.

SNPs in the D-AKAP2 gene have been identified herein that are associated with morbidity using a genome-wide association study from an age-stratified healthy population and 6,500 gene-based SNPs. The combined genetic and biochemical evidence points to the Ile/Val variant as the functional polymorphism. The Val variant is contemplated to be the deleterious allele in a Caucasian-American cohort, and this finding is replicated in Hispanic-Americans. The variant maps to the conserved AKB domain of the D-AKAP2 gene. It has been found that the I646V variation impacts the binding to PKA in an isoform-specific manner both in vitro and in cells. The lie variant have been found to bind three-fold weaker to the RIa isoform than to the Val variant. At the cellular level, this affinity difference results in a dramatic decrease in compartmentalization of RIa for the Ile variant. The Ile/Val variant has been found to be critical for binding to only the RIa isoform of PKA.

Increasing evidence suggests that the RI and RII isoforms of PKA have distinct functions. The RI isoform has been implicated in a variety of biological functions such as cell proliferation, tumor suppression, immune regulation, and embryonic development. In addition, the RIa isoform plays a significant role in maintaining cAMP-regulation of PKA as evidenced by the embryonic lethality of mice deficient in the gene. Interestingly, these mice have defects in cardiac morphogenesis.

The identified correlation of the I646V variant (e.g., -G- at position 2073 of SEQ ID NO:1) with the EKG PR interval measurement is contemplated herein to indicate that the I646V polymorphism is a predisposing factor for a disease or disorder, such as a cardiovascular disease or disorder manifesting a cardiac phenotype. For example, individuals homozygous for the Val variant exhibit shorter depolarization intervals of the atrium (PR) as compared to individuals homozygous for Ile. This phenotypic correlation combined with reports in the literature supporting a role for AKAP mediated PKA signaling in normal cardiac function suggest a lead into the pathogenesis coded for by this functional variant. AKAP-mediated targeting of PKA in cardiac myocytes has been implicated in regulating cell contractility (Fink et al., Cir. Res., 88:291-297, 2001). Stimulation of the β-adrenergic signaling pathway in cardiac myocytes results in activation of PKA and phosphorylation of a variety of PKA substrates, including the sarcolemmal L-type Ca2+ channel, the ryanodine receptor (RyR), phospholamban (PLB) of the sarcoplasmic reticulum (SR), the myofibrillar proteins troponin I (TnI) and myosin binding protein C (MBP-C) (Holroyde et al., Biochim. Biophys. Acta, 586:63-69, 1979; Kranias et al., Nature, 298:182-184, 1982; Brum et al., Pflugers Arch., 401:111-118, 1984; Garvey et al., Biochem. J., 249:709-714, 1988; Marx et al., Cell, 101:365-376, 1999). Phosphorylation of these substrates acts in concert to generate both enhanced contractility and accelerated relaxation in response to β-adrenergic stimulation.

Although PKA has broad substrate specificity, it can be highly selective by targeting of PKA to distinct subcellular locations via interaction with AKAPs (Colledge et al., Trends Cell Biol., 19:216-221, 1999). Three AKAPs have been shown to interact with PKA in cardiac myocytes, muscle-selective AKAP (mAKAP), AKAP18 and Yotiao. mAKAP targets PKA to the perinuclear region of differentiated myocytes, coordinating both PKA and phosphodiesterase activity in a single complex (Kapiloff et al., J. Cell Sci., 114:3167-3176, 2001). AKAP18 couples PKA to L-type Ca2+ channels, which enhances Ca2+ influx through the channel following β-adrenergic stimulation (Gray et al., J. Biol. Chem., 272:6297-6302, 1997). Yotiao, previously associated with NMDA receptors, has been shown to interact with the KCNQ1-KCNE1 K+ channel subunits in human hearts (Marx et al., Science, 295:496-499, 2002). This channel is responsible for the slow delayed rectifier current that repolarizes the myocyte membrane and controls action potential duration. Mutations in this channel associated with hereditary long QT syndrome abolish Yotiao interactions with the channel, thereby attenuating PKA regulation (Marx et al., 2002). Additionally, in cardiac myocytes the RIα subunit is the predominant isoform associated with the sarcolemma (Robinson et al., Arch. Biochem. Biophys., 330:181-187, 1996; Reinitz et al., Arch. Biochem. Biophys., 348:391-402, 1997). Mutations in RI are associated with both familial cardiac myxomas and Carney complex, implicating this isoform in cardiac growth and differentiation (Casey et al., J. Clin. Invest., 106:R31-38, 2000; Kirschner et al., Nat. Genet., 26:89-92, 2000). Direct involvement of PKA in heart disease was also recently reported in a transgenic mice study (Antos et al., Circ. Res., 89:997-1004, 2001). The transgenic mice that overexpressed the catalytic subunit of PKA developed dilated cardiomyopathy with reduced cardiac contractility and increased risk of arrhythmias. These cardiac abnormalities correlated with PKA-mediated hyperphosphorylation of the ryanodine receptor and Ca2+ release from the sarcoplasmic reticulum (SR) and phospholamban, which regulates the activity of the SR Ca2+ -ATPase (Antos et al., 2001).

D-AKAP2 (AKAP10-5) contains a PDZ binding motif (TKL) at the C-terminus (FIG. 3a), which is contemplated herein to serve as a targeting domain to membrane-bound receptors or ion-channels (Harris et al., J. Cell Sci., 114:3219-3231, 2001), and two RGS domains, which are contemplated herein to coordinate upstream G alpha signaling with downstream PKA signaling. It is contemplated herein that D-AKAP2 is part of a signaling complex associated with a cardiac ion-channel. The D-AKAP2 variants is contemplated herein to impact the phosphorylation state of the ion-channel by recruiting different amounts of PKA-RIa and thereby modulate heart contraction. This model is in agreement with the observed association with an EKG phenotype. The shorter depolarization intervals for Val/Val homozygous individuals is contemplated herein to be due to increased activation of ion-channels in cardiac myocytes.

In summary, through the analysis of significant morbidity markers in a well-phenotyped healthy twin population, cardiac phenotypes have been associated to particular D-AKAP2 genotypes.

1. Method for Indicating Increased Susceptibility of a Subject to a Disease or Disorder

Accordingly, provided herein are methods for indicating increased susceptibility of a subject to a disease or disorder, comprising:

conducting an EKG examination;

determining the EKG-PR-interval in the subject, wherein, if the EKG-PR-interval is decreased, then

determining the amino acid present in the subject at position 646 of AKAP10/D-AKAP2 (SEQ ID NO:2) or the nucleotide present at position corresponding to nucleotide 2073 of SEQ ID NO:1, wherein the presence of Val at position 646 of SEQ ID NO:2 or the presence of a -G- at nucleotide position 2073 of SEQ ID NO:1, indicates increased susceptibility to a disease or disorder. The disease or disorder can be selected from among cardiovascular disorders, cardiac disease, proliferative disorders, neurological disorders, neurodegenerative disorders, obesity, diabetes and peripheral retinopathies.

In one embodiment, the EKG-PR-interval in the subject is compared to a predetermined age-matched standard EKG-PR-interval. The predetermined standard EKG-PR-interval can be obtained from a known age-matched control group that is homozygous -AA- at a position corresponding to nucleotide 2073 of SEQ ID NO:1 or homozygous Ile/Ile at a position corresponding to position 646 of SEQ ID NO:2. The predetermined standard EKG-PR-interval can be obtained from a known age-matched control group that is heterozygous -GA- at a position corresponding to nucleotide 2073 of SEQ ID NO:1 or heterozygous Val/Ile at a position corresponding to position 646 of SEQ ID NO:2. The predetermined standard EKG-PR-interval can be obtained from a known age-matched control group that is selected from either homozygous -AA- at a position corresponding to nucleotide 2073 of SEQ ID NO:1 or homozygous Ile/Ile at a position corresponding to position 646 of SEQ ID NO:2; or heterozygous -GA- at a position corresponding to nucleotide 2073 of SEQ ID NO:1 or heterozygous Val/Ile at a position corresponding to position 646 of SEQ ID NO:2. In another embodiment, the predetermined standard EKG-PR-interval is obtained from a control age-matched subject without heart disease.

In one embodiment, the subject is heterozygous -GA- at a position corresponding to nucleotide 2073 of SEQ ID NO:1 or heterozygous Val/Ile at a position corresponding to position 646 of SEQ ID NO:2. In another embodiment, the subject is homozygous -GG- at a position corresponding to nucleotide 2073 of SEQ ID NO:1 or homozygous Val/Val at a position corresponding to position 646 of SEQ ID NO:2.

Also provided are methods for indicating increased susceptibility of a subject to a disease or disorder associated with the cardiovascular system, comprising:

conducting an EKG exam;

determining the EKG-PR-interval in the subject, wherein, if the EKG-PR-interval is decreased, then

determining the amino acid present at position 646 of AKAP10/D-AKAP2 (SEQ ID NO:2) or the nucleotide present at position corresponding to nucleotide 2073 of SEQ ID NO:1, wherein the presence of Val at position 646 of SEQ ID NO:2 or the presence of a -G- at nucleotide position 2073 of SEQ ID NO:1, indicates increased susceptibility to a disease or disorder associated with the cardiovascular system. The EKG-PR-interval in the subject can be compared to a predetermined age-matched standard EKG-PR-interval to determine whether it is decreased. The predetermined standard EKG-PR-interval can be obtained from a known age-matched control that is homozygous -AA- at a position corresponding to nucleotide 2073 of SEQ ID NO:1 or homozygous Ile/Ile at a position corresponding to position 646 of SEQ ID NO:2. The predetermined standard EKG-PR-interval can be obtained from a known age-matched control group that is heterozygous -GA- at a position corresponding to nucleotide 2073 of SEQ ID NO:1 or heterozygous Val/Ile at a position corresponding to position 646 of SEQ ID NO:2. The predetermined standard EKG-PR-interval can be obtained from a known age-matched control group that is selected from either homozygous -AA- at a position corresponding to nucleotide 2073 of SEQ ID NO:1 or homozygous Ile/Ile at a position corresponding to position 646 of SEQ ID NO:2; or heterozygous -GA- at a position corresponding to nucleotide 2073 of SEQ ID NO:1 or heterozygous Val/Ile at a position corresponding to position 646 of SEQ ID NO:2. In another embodiment, the predetermined standard EKG-PR-interval can be obtained from a control age-matched subject without heart disease.

In particular embodiments of the various methods provided herein, in the context of determining whether the EKG-PR-interval is decreased, a decreased EKG-PR-interval is less than 150 for a subject 40 or more year old. In another embodiment, a decreased EKG-PR-interval is less than 155 for a subject 50 or more year old. In another embodiment, a decreased EKG-PR-interval is less than 150 for a subject 50 or more year old. In another embodiment, a decreased EKG-PR-interval is less than 160 for a subject 60 or more year old. In another embodiment, a decreased EKG-PR-interval is less than 155 for a subject 60 or more year old. In another embodiment, a decreased EKG-PR-interval is less than 150 for a subject 60 or more year old.

In yet other embodiments, a decreased EKG-PR-interval for the subject is less than 146. In another embodiment, a decreased EKG-PR-interval for the subject is less than 130. In another embodiment, a decreased EKG-PR-interval for the subject is less than 120.

The disease or disorder can be selected from one or more of the group consisting of: atrial fibrillation, sick sinus syndrome, sudden cardiac arrest, ventricular arrythmia, ventricular fibrillation, ventricular tachycardia, Wolf-Parkinson-White (WPW) Syndrome, Lown-Ganong-Levin (LGL) Syndrome, hypertension. In one embodiment, the methods can further comprise monitoring the subject for cardiovascular disease.

In one embodiment, the subject is heterozygous -GA- at a position corresponding to nucleotide 2073 of SEQ ID NO:1 or heterozygous Val/Ile at a position corresponding to position 646 of SEQ ID NO:2. In another embodiment, the subject is homozygous -GG- at a position corresponding to nucleotide 2073 of SEQ ID NO:1 or homozygous Val/Val at a position corresponding to position 646 of SEQ ID NO:2.

In another embodiment, the methods can further comprise administering to the subject prophylactic steps. For example, in view of developments in genetics and technology as well as epidemiology, the methods provided herein permit the determination of the probability and risk assessment for the development of disease, in particular heart disease in an individual. Using the genetic screening methods herein and/or family health histories, it is possible to predict the probability a particular individual has for developing any one of several types of disease, such as heart disease. Those individuals identified as being predisposed to developing a particular form of disease by using the methods provided herein, can take prophylactic steps towards reducing the risk of the particular disease, such as a heart disease. Accordingly, high-risk individuals identified herein can take one or more of the well-known prophylactic steps against the form of disease that they have a predisposition to develop.

a. Methods of Assessing the Susceptibility of a Subject to a Disease or Disorder Associated with the Cardiovascular System

Also provided herein are methods of assessing the susceptibility of a subject to a disease or disorder associated with the cardiovascular system, the method comprising determining the amino acid at position 646 of AKAP10/D-AKAP2 (SEQ ID NO:2) or the nucleotide present at position corresponding to nucleotide 2073 of SEQ ID NO: 1, wherein the presence of Val at position 646 of SEQ ID NO:2 or the presence of a -G- at nucleotide position 2073 of SEQ ID NO:1, indicates increased susceptibility to a disease or disorder associated with the cardiovascular system.

Also provided herein are methods of diagnosing a disease or disorder associated with the cardiovascular system, comprising detecting the presence of Val at 646 of D-AKAP2 (SEQ ID NO:2) or the presence of a G at a nucleotide position corresponding to nucleotide 2073 of SEQ ID NO:1, wherein the presence of Val at position 646 of SEQ ID NO:2 or the presence of a -G- at nucleotide position 2073 of SEQ ID NO:1, indicates the presence of a disease or disorder associated with the cardiovascular system. In these methods, the disease or disorder can be from among one or more of the group consisting of: atrial fibrillation, sick sinus syndrome, sudden cardiac arrest, ventricular arrythmia, ventricular fibrillation, ventricular tachycardia, Wolf-Parkinson-White (WPW) Syndrome, Lown-Ganong-Levin (LGL) Syndrome, hypertension.

In one embodiment, the subject is heterozygous -GA- at a position corresponding to nucleotide 2073 of SEQ ID NO:1 or heterozygous Val/Ile at a position corresponding to position 646 of SEQ ID NO:2. In another embodiment, the subject is homozygous -GG- at a position corresponding to nucleotide 2073 of SEQ ID NO:1 or homozygous Val/Val at a position corresponding to position 646 of SEQ ID NO:2.

2. Methods for Determining Responsiveness of a Subject to One or More β-Blocking Agents

The presence of the I646V variant in a subject is contemplated herein to affect the D-AKAP2-mediated β-adrenergic signaling pathway. For example, it is contemplated herein that the presence of 1 or 2 copies of the I646V variant (e.g., a -g- at nucleotide 2073 of SEQ ID NO:1) in a subject renders the subject resistant to treatment with, or the effects of, the well-known β-blockers. Beta-blockers (β-adrenergic blocking drugs) “block” the effects of adrenaline on the body's beta receptors. This slows the nerve impulses that travel through the heart. As a result, the heart does not have to work as hard because it needs less blood and oxygen. Beta-blockers also block the impulses that can cause an arrhythmia. In one embodiment, the heterozygous presence of the I646V variant (e.g., a -GA- heterozygous genotpye at a nucleotide corresponding to position 2073 of SEQ ID NO:1) is contemplated herein to produce a β-blocker resistance phenotype. In another embodiment, the homozygous presence of the I646V variant (e.g., a -GG- heterozygous genotpye at a nucleotide corresponding to position 2073 of SEQ ID NO:1) is contemplated herein to produce the β-blocker resistance phenotype.

Accordingly, provided herein are methods for determining responsiveness of a subject to one or more β-blocking agents, comprising:

detecting for the subject the presence or absence of Val at position 646 of SEQ ID NO:2 or a -G- nucleotide at a position corresponding to position 2073 of SEQ ID NO: 1, wherein the presence of a Val at position 646 of SEQ ID NO:2 or a -G- at nucleotide 2073 of SEQ ID NO: 1, is indicative of an increased likelihood that a subject has a modulated response to one or more β-blocking agents compared to a subject who does not have the allelic variant. In one embodiment, the modulated response is a decreased response to one or more β-blocking agents compared to a subject who does not have the allelic variant. In another embodiment, the decreased response is a non-response to one or more β-blocking agents compared to a subject who does not have the allelic variant. In yet another embodiment, the modulated response is an increased response to one or more β-blocking agents compared to a subject who does not have the allelic variant. The β-blocker ican bean antagonist of a β-adrenergic receptor. In another embodiment, the β-blocker is an agonist of a β-adrenergic receptor.

Also provided herein are methods for determining responsiveness of a subject to one or more β-blocking agents, comprising:

detecting the presence or absence of Val at position 646 of SEQ ID NO:2 or a -G- nucleotide at a position corresponding to position 2073 of SEQ ID NO: 1, wherein the presence of a Val at position 646 of SEQ ID NO:2 or a -G- at nucleotide 2073 of SEQ ID NO:1, is indicative of an increased likelihood that a subject has an increased response to one or more β-blocking agents compared to a subject who does not have the allelic variant. In one embodiment, the β-blocker is an antagonist of a β-adrenergic receptor. In another embodiment, the β-blocker is an agonist of a β-adrenergic receptor.

Also provided herein, are methods for determining responsiveness of a subject to one or more β-blocking agents, comprising:

detecting for the subject the presence or absence of Val at position 646 of SEQ ID NO:2 or a -G- nucleotide at a position corresponding to position 2073 of SEQ ID NO: 1, wherein the presence of a Val at position 646 of SEQ ID NO:2 or a -G- at nucleotide 2073 of SEQ ID NO:1, is indicative of an increased likelihood that a subject is non-responsive to one or more β-blocking agents compared to a subject who does not have the allelic variant. In one embodiment, the β-blocker is an antagonist of a β-adrenergic receptor. In another embodiment, the β-blocker is an agonist of a β-adrenergic receptor.

Also provided herein are methods for determining responsiveness of a subject to one or more β-blocking agents, comprising:

detecting the presence or absence of Val at position 646 of SEQ ID NO:2 or a -G- nucleotide at a position corresponding to position 2073 of SEQ ID NO: 1, wherein the presence of a Val at position 646 of SEQ ID NO:2 or a -G- at nucleotide 2073 of SEQ ID NO:1, is indicative of an increased likelihood that a subject is hyper-responsive to one or more β-blocking agents compared to a subject who does not have the allelic variant. The β-blocker can be an antagonist of a β-adrenergic receptor. In another embodiment, the β-blocker is an agonist of a β-adrenergic receptor.

Exemplary β-blockers well known in the art include, but are not limited to, Acebutolol, atenolol, Betaxolol, Bisoprolol, Carteolol, Carbedilol, Esmolol, Labetolol, Metoprolol, Nadolol, Penbutolol, Pindolol, Propranolol and Timolol. These β-blocking agents are used to treat: high blood pressure, angina, abnormal heart rythms, hypertrophic cardiomyopathy, heart failure, vasovagal fainting, migraines, essential tremor, bleeding from esophageal varices, stage fright, glaucoma and to prolong survival of heart attack patients. Thus, the methods provided herein are useful to identify subjects that require or would benefit from a different treatment regimen than the use of β-blockers for their respective disease or disorder.

In one embodiment, the subject is heterozygous -GA- at a position corresponding to nucleotide 2073 of SEQ ID NO:1 or heterozygous Val/Ile at a position corresponding to position 646 of SEQ ID NO:2. In another embodiment, the subject is homozygous -GG- at a position corresponding to nucleotide 2073 of SEQ ID NO:1 or homozygous Val/Val at a position corresponding to position 646 of SEQ ID NO:2.

3. Method for Indicating Susceptibility of a Subject to Acquired Long Q-T Syndrome

The long QT syndrome (LQTS) is an abnormality of the heart's electrical system. The mechanical function of the heart is entirely normal. The electrical problem is due to defects in heart muscle cell structures called ion channels. These electrical defects predispose affected persons to a very fast heart rhythm (arrhythmia) called torsade de pointes which leads to sudden loss of consciousness (syncope) and can cause sudden cardiac death. The syndrome can be inherited (the genetic form) or acquired. The inherited long QT Syndrome was first clearly described in 1957. There are two variants, the autosomal dominant Romano-Ward type and the autosomal recessive Jervell and Lange Nielsen type. Even though LQTS was described almost 40 years ago, too many physicians are unaware of it. Whereas acquired long QT syndrome is most often due to the administration of medication. These medications are contraindicated in patients with the long QT syndrome.

There are a number of drugs which are known to prolong the QT-interval and to cause heart rhythm abnormalities, particularly in patients with the long QT syndrome (LQTS). Accordingly, patients with LQTS should always inquire their physician or other health care provider about the risk of any medication suggested or prescribed for them. In addition, LQTS-patients should always inform their doctor(s) and dentist(s) about their disease and make sure they know there are many medications which are contraindicated in this condition. For example, the department of pharmacology at Georgetown university provides a complete and up-to-date list of drugs that prolong the QT-interval (external link).

The frequency is unknown but long Q-T syndrome appears to be a common cause of sudden and unexplained death in children and young adults. It is certainly much more common than previously thought. It can be as frequent as 1 in 5,000, and can cause 3,000-4,000 sudden deaths in children and young adults each year in the United States. The Jervell and Lange Nielsen form is associated with congenital deafness and is rare, but the Romano-Ward variant, with normal hearing, is being recognized with increasing frequency.

The usual symptoms are syncope (sudden loss of consciousness) or sudden death, typically occurring during physical activity or emotional upset. These most commonly begin in preteen to teenage years, but can present from a few days of age to middle age. The syncopal episodes are often misdiagnosed as the common faint (vasovagal event) or a seizure. Actual seizures are uncommon in long QT syndrome, but epilepsy is one of the common errors in diagnosis. Sudden loss of consciousness during physical exertion or during emotional excitement should strongly raise the possibility of the long QT syndrome. A family history of unexplained syncope or sudden death in young people should also raise suspicion. Importantly, about one third of individuals who have the long QT syndrome never exhibit symptoms, and therefore, the lack of symptoms does not exclude a person or family from having LQTS. Any young person that has an unexplained cardiac arrest should be considered for LQTS, as well as those with unexplained syncope.

Since the electrocardiographic Q-T intervals vary in a given individual from day to day, and since Q-T prolongation in affected individuals can be mild, the diagnosis can be missed even if an EKG is performed. Therefore, because a patients can not receive an existing effective treatment in time because the condition which can kill quickly is sometimes very hard to diagnose, other methods of diagnosing long Q-T or a predisposition for long Q-T syndrome would be very useful. It is contemplated herein that the I646V variant in D-AKAP2 is indicative of a predisposition to the acquired form of Long Q-T syndrome. Accordingly, provided herein are methods that identify subjects who are predisposed or susceptible to acquired long Q-T syndrome. These methods are useful in identifying the class of subjects who should avoid taking particular medications, such as the well-documented group of medications that those diagnosed with long Q-T syndrome should avoid.

For example, provided herein are methods for indicating susceptibility of a subject to acquired long Q-T syndrome, comprising:

detecting the presence or absence of Val at position 646 of SEQ ID NO:2 or presence or absence of a -G- nucleotide at a position corresponding to position 2073 of SEQ ID NO: 1, wherein the presence of a Val at position 646 of SEQ ID NO:2 or a -G- at nucleotide 2073 of SEQ ID NO:1, is indicative of increased susceptibility to acquired long Q-T syndrome, compared to the susceptibility of a subject who does not have the allelic variant. The detecting step can be effected by a method selected from the group consisting of allele specific hybridization, primer specific extension, oligonucleotide ligation assay, restriction enzyme site analysis and single-stranded conformation polymorphism analysis. In addition, the detecting step can comprise mass spectrometry. The detection can be effected by detecting a signal moiety selected from the group consisting of radioisotopes, enzymes, antigens, antibodies, spectrophotometric reagents, chemiluminescent reagents, fluorescent reagents and other light producing reagents.

In one embodiment, the subject is heterozygous -GA- at a position corresponding to nucleotide 2073 of SEQ ID NO:1 or heterozygous Val/Ile at a position corresponding to position 646 of SEQ ID NO:2. In another embodiment, the subject is homozygous -GG- at a position corresponding to nucleotide 2073 of SEQ ID NO:1 or homozygous Val/Val at a position corresponding to position 646 of SEQ ID NO:2.

4. Methods for Indicating Susceptibility to Morbidity, Increased or Early Mortality, or Morbidity and Increased or Early Mortality of a Subject

Also provided herein, are methods for indicating susceptibility to morbidity, increased or early mortality, or morbidity and increased or early mortality of a subject; comprising:

conducting an EKG exam;

determining the EKG-PR-interval in the subject, wherein if the EKG-PR-interval is decreased; then

determining the amino acid at position 646 of AKAP10/D-AKAP2 (SEQ ID NO:2) or the nucleotide present at position corresponding to nucleotide 2073 of SEQ ID NO:1, wherein the presence of Val at position 646 of SEQ ID NO:2 or the presence of a -G- at nucleotide position 2073 of SEQ ID NO: 1, indicates increased susceptibility to morbidity, increased or early mortality, or morbidity and increased or early mortality of a subject. The EKG-PR-interval in the subject can be compared to a predetermined standard EKG-PR-interval. The predetermined standard EKG-PR-interval can be obtained from a known age-matched control that is homozygous -AA- at a position corresponding to nucleotide 2073 of SEQ ID NO:1 or homozygous Ile/Ile at a position corresponding to position 646 of SEQ ID NO:2. The detecting step can be effected by a method selected from the group consisting of allele specific hybridization, primer specific extension, oligonucleotide ligation assay, restriction enzyme site analysis and single-stranded conformation polymorphism analysis. The detecting step can comprise mass spectrometry. The detection step can be effected by detecting a signal moiety selected from the group consisting of radioisotopes, enzymes, antigens, antibodies, spectrophotometric reagents, chemiluminescent reagents, fluorescent reagents and other light producing reagents.

In one embodiment, the subject is heterozygous -GA- at a position corresponding to nucleotide 2073 of SEQ ID NO:1 or heterozygous Val/Ile at a position corresponding to position 646 of SEQ ID NO:2. In another embodiment, the subject is homozygous -GG- at a position corresponding to nucleotide 2073 of SEQ ID NO:1 or homozygous Val/Val at a position corresponding to position 646 of SEQ ID NO:2.

D. Detection of Polymorphisms in Human AKAP10 Genes

Methods of determining the presence or absence of allelic variants of a human AKAP10 gene are also provided. In particular methods, the detection or identification of a G, C, or T nucleotide at position 2073 of the sense strand of the human AKAP10 gene coding sequence (see SEQ ID NO: 1), or the detection or identification of a C, G or A nucleotide at the same position in the antisense strand of the human AKAP10 gene coding sequence, indicates the presence of an allelic variant. In these particular methods, the detection or identification of an A nucleotide at position 2073 of the sense strand of the human AKAP10 gene coding sequence, or the detection or identification of a T nucleotide at the same position in the antisense strand of the human AKAP10 gene coding sequence, indicates the absence of polymorphism.

Other methods for determining the presence or absence of an allelic variant of the AKAP10 gene detect or identify a nucleotide other than a C at position 83587 of the SEQ ID NO: 17 or a nucleotide other than a G on the complementary strand, a nucleotide other than a G at position 129600 of the SEQ ID NO: 17 or a nucleotide other than a C on the complementary strand or a nucleotide other than T at position 156,277 of SEQ ID NO: 17 or a nucleotide other than A on the complementary strand.

1. Nucleic Acid Detection Methods

Generally, these methods are based in sequence-specific polynucleotides, oligonucleotides, probes and primers. Any method known to those of skill in the art for detecting a specific nucleotide within a nucleic acid sequence or for determining the identity of a specific nucleotide in a nucleic acid sequence is applicable to the methods of determining the presence or absence of an allelic variant of the AKAP10 gene. Such methods include, but are not limited to, techniques utilizing nucleic acid hybridization of sequence-specific probes, nucleic acid sequencing, selective amplification, analysis of restriction enzyme digests of the nucleic acid, cleavage of mismatched heteroduplexes of nucleic acid and probe, alterations of electrophoretic mobility, primer specific extension, oligonucleotide ligation assay and single-stranded conformation polymorphism analysis. In particular, primer extension reactions that specifically terminate by incorporating a dideoxynucleotide are useful for detection. Several such general nucleic acid detection assays are known (see, e.g., U.S. Pat. No. 6,030,778).

a. Primer Extension-Based Methods

Several primer extension-based methods for determining the identity of a particular nucleotide in a nucleic acid sequence have been reported (see, e.g., PCT Application Nos. PCT/US96/03651 (WO96/29431), PCT/US97/20444 (WO 98/20166), PCT/US97/20194 (WO 98/20019), PCT/US91/00046 (WO91/13075), and U.S. Pat. Nos. 5,547,835, 5,605,798, 5,622,824, 5,691,141, 5,872,003, 5,851,765, 5,856,092, 5,900,481, 6,043,031, 6,133,436 and 6,197,498.) In general, a primer is prepared that specifically hybridizes adjacent to a polymorphic site in a particular nucleic acid molecule. The primer is then extended in the presence of one or more dideoxynucleotides, typically with at least one of the dideoxynucleotides being the complement of the nucleotide that is polymorphic at the site. The primer and/or the dideoxynucleotides can be labeled to facilitate a determination of primer extension and identity of the extended nucleotide.

In an exemplary method, primer extension and/or the identity of the extended nucleotide(s) are determined by mass spectrometry (see, e.g., PCT Application Nos. PCT/US96/03651 (WO96/29431), PCT Application No. PCT/US97/20444 (WO 98/20166), PCT Application No. PCT/US97/20194 (WO 98/20019), PCT Application No. PCT/US91/00046 (WO91/13075), and U.S. Pat. Nos. 5,605,798, 5,622,824, 5,856,092.

b. Polymorphism-Specific Probe Hybridization

A typical detection method is allele specific hybridization using probes overlapping the polymorphic site and having about 5, 10, 15, 20, 25, or 30 nucleotides around the polymorphic region. The probes can contain naturally occurring or modified nucleotides (see U.S. Pat. No. 6,156,501). For example, oligonucleotide probes can be prepared in which the known polymorphic nucleotide is placed centrally (allele-specific probes) and then hybridized to target DNA under conditions which permit hybridization only if a perfect match is found (Saiki et al. (1986) Nature 324:163; Saiki et al. (1989) Proc. Natl. Acad. Sci USA 86:6230; and Wallace et al. (1979) Nucl. Acids Res. 6:3543). Such allele specific oligonucleotide hybridization techniques can be used for the simultaneous detection of several nucleotide changes in different polymorphic regions. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid. In one embodiment, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix, Santa Clara, Calif.). Mutation detection analysis using these chips comprising oligonucleotides, also termed “DNA probe arrays” is described e.g., in Cronin et al. (1996) Human Mutation 7:244 and in Kozal et al. (1996) Nature Medicine 2:753. In one embodiment, a chip includes all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

c. Nucleic Acid Amplification-Based Methods

In other detection methods, it is necessary to first amplify at least a portion of an AKAP gene prior to identifying the allelic variant. Amplification can be performed, e.g., by PCR and/or LCR, according to methods known in the art. In one embodiment, genomic DNA of a cell is exposed to two PCR primers and amplification is performed for a number of cycles sufficient to produce the required amount of amplified DNA. In typical embodiments, the primers are located between 150 and 350 base pairs apart.

Alternative amplification methods include: self sustained sequence replication (Guatelli, J. C. et al., 1990, Proc. Natl. Acad. Sci. U.S.A. 87:1874-1878), transcriptional amplification system (Kwoh, D. Y. et al., 1989, Proc. Natl. Acad. Sci. U.S.A. 86:1173-1177), Q-Beta Replicase Lizardi, P. M. et al., 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

Alternatively, allele specific amplification technology, which depends on selective PCR amplification can be used in conjunction with the alleles provided herein. Oligonucleotides used as primers for specific amplification can carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1 993) Tibtech 11:238; Newton et al. (1989) Nucl. Acids Res. 17:2503). In addition it can be desirable to introduce a restriction site in the region of the mutation to create cleavage-based detection (Gasparini et al. (1992) Mol. Cell Probes 6:1).

d. Nucleic Acid Sequencing-Based Methods

In one embodiment, any of a variety of sequencing reactions known in the art can be used to directly sequence at least a portion of an AKAP gene and to detect allelic variants, e.g., mutations, by comparing the sequence of the sample sequence with the corresponding wild-type (control) sequence. Exemplary sequencing reactions include those based on techniques developed by Maxam and Gilbert (Proc. Natl. Acad. Sci. USA (1977) 74:560) or Sanger (Sanger et al. (1977) Proc. Natl. Acad. Sci 74:5463). It is also contemplated that any of a variety of automated sequencing procedures can be used when performing the subject assays (Biotechniques (1995) 19:448), including sequencing by mass spectrometry (see, for example, U.S. Pat. Nos. 5,547,835, 5,691,141, and International PCT Application No. PCT/US94/00193 (WO 94/16101), entitled “DNA Sequencing by Mass Spectrometry” by H. Koster; U.S. Pat. Nos. 5,547,835, 5,622,824, 5,851,765, 5,872,003, 6,074,823, 6,140,053 and International PCT Application No. PCT/US94/02938 (WO 94/21822), entitled “DNA Sequencing by Mass Spectrometry Via Exonuclease Degradation” by H. Koster), and U.S. Pat. Nos. 5,605,798, 6,043,031, 6,197,498, and International Patent Application No. PCT/US96/03651 (WO 96/29431) entitled “DNA Diagnostics Based on Mass Spectrometry” by H. Koster; Cohen et al. (1996) Adv Chromatography 36:127-162; and Griffin et al. (1993) Appl. Biochem Biotechnol 38:147-159). It will be evident to one skilled in the art that, for certain embodiments, the occurrence of only one, two or three of the nucleic acid bases need be determined in the sequencing reaction. For instance, A-track sequencing or an equivalent, e.g., where only one nucleotide is detected, can be carried out. Other sequencing methods are known (see, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”).

e. Restriction Enzyme Digest Analysis

In some cases, the presence of a specific allele in nucleic acid, particularly DNA, from a subject can be shown by restriction enzyme analysis. For example, a specific nucleotide polymorphism can result in a nucleotide sequence containing a restriction site which is absent from the nucleotide sequence of another allelic variant.

f. Mismatch Cleavage

Protection from cleavage agents, such as, but not limited to, a nuclease, hydroxylamine or osmium tetroxide and with piperidine, can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of an allelic variant with a sample nucleic acid, e.g, RNA or DNA, obtained from a tissue sample. The double-stranded duplexes are treated with an agent, which cleaves single-stranded regions of the duplex such as duplexes formed based on basepair mismatches between the control and sample strands. For instance, RNA/DNA duplexes can be treated with RNase and DNA/DNA hybrids treated with SI nuclease to enzymatically digest the mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they differ (see, for example, Cotton et al. (1988) Proc. Natl. Acad Sci USA 85:4397; Saleeba et al. (1992) Methods Enzymod. 217:286-295). The control or sample nucleic acid is labeled for detection.

g. Electrophoretic Mobility Alterations

In other embodiments, alteration in electrophoretic mobility is used to identify the type of allelic variant in an AKAP gene. For example, single-strand conformation polymorphism (SSCP) can be used to detect differences in electrophoretic mobility between mutant and wild type nucleic acids (Orita et al. (1989) Proc. Natl. Acad. Sci. USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In another embodiment, the subject method uses heteroduplex analysis to separate double stranded heteroduplex molecules on the basis of changes in electrophoretic mobility (Keen et al. (1991) Trends Genet 7:5).

h. Polyacrylamide Gel Electrophoresis

In yet another embodiment, the identity of an allelic variant of a polymorphic region of an AKAP gene is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

i. Oligonucleotide Ligation Assay (OLA)

In another embodiment, identification of the allelic variant is carried out using an oligonucleotide ligation assay (OLA), as described, e.g., in U.S. Pat. No. 4,998,617 and in Landegren, U. et al., Science 241:1077-1080 (1988). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA, which is then detected using OLA.

Several techniques based on this OLA method have been developed and can be used to detect specific allelic variants of a polymorphic region of a gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucl. Acids Res. 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

j. SNP Detection Methods

Also provided are methods for detecting single nucleotide polymorphisms. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment, a solution-based method for determining the identity of the nucleotide of a polymorphic site is employed (Cohen, D. et al. (French Patent 2,650,840; PCT Application No. WO91/02087)). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

k. Genetic Bit Analysis

An alternative method, known as Genetic Bit Analysis or GBA™ is described by Goelet, et al. (U.S. Pat. No. 6,004,744, PCT Application No. 92/15712). The method of Goelet, et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Application No. WO91/02087), the method of Goelet, et al. is typically a heterogeneous phase assay, in which the primer or the target molecule is immobilized to a solid phase.

l. Other Primer-Guided Nucleotide Incorporation Procedures

Other primer-guided nucleotide incorporation procedures for assaying polymorphic sites in DNA have been described (Komher, J. S. et al., Nucl. Acids Res. 17:7779-7784 (1989); Sokolov, B. P., Nucl. Acids Res. 18:3671 (1990); Syvanen, A. C., et al., Genomics 8:684-692 (1990), Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. C., et al., Amer. J. Hum. Genet. 52:46-59 (1993)).

For determining the identity of the allelic variant of a polymorphic region located in the coding region of a gene, yet other methods than those described above can be used. For example, identification of an allelic variant which encodes a mutated protein can be performed by using an antibody specifically recognizing the mutant protein in, e.g., immunohistochemistry or immunoprecipitation. Binding assays are known in the art and involve, e.g., obtaining cells from a subject, and performing binding experiments with a labeled lipid, to determine whether binding to the mutated form of the protein differs from binding to the wild-type protein.

m. Molecular Structure Determination

If a polymorphic region is located in an exon, either in a coding or non-coding region of the gene, the identity of the allelic variant can be determined by determining the molecular structure of the mRNA, pre-mRNA, or cDNA. The molecular structure can be determined using any of the above described methods for determining the molecular structure of the genomic DNA, e.g., sequencing and SSCP.

n. Mass Spectrometric Methods

Nucleic acids can also be analyzed by detection methods and protocols, particularly those that rely on mass spectrometry (see, e.g., U.S. Pat. Nos. 5,605,798, 6,043,031, 6,197,498, and International Patent Application No. WO 96/29431, allowed co-pending U.S. application Ser. No. 08/617,256, allowed co-pending U.S. application Ser. No. 08/744,481, U.S. application Ser. No. 08/990,851, International PCT Application No. WO 98/20019). These methods can be automated (see, e.g., co-pending U.S. application Ser. No. 09/285,481, which describes an automated process line). Among the methods of analysis herein are those involving the primer oligo base extension (PROBE) reaction with mass spectrometry for detection (see e.g., U.S. Pat. Nos. 6,043,031 and 6,197,498, patent application Ser. Nos. 09/287,681, 09/287,682, and 09/287,679, allowed co-pending U.S. application Ser. No. 08/744,481, International PCT Application No. PCT/US97/20444 (WO 98/20166), and based upon U.S. Pat. Nos. 5,900,481, 6,024,925, 6,074,823, application Ser. Nos. 08/746,055, 08/786,988, 08/933,792, 08/746,055, and 08/786,988; see, also U.S. application Ser. No. 09/074,936, and published International PCT Application No. PCT/US97/20195 (WO 98/20020)).

A typical format for performing the analyses is a chip based format in which the biopolymer is linked to a solid support, such as a silicon or silicon-coated substrate, for example, in the form of an array. More typically, when analyses are performed using mass spectrometry, particularly MALDI, nanoliter volumes of sample are loaded on, such that the resulting spot is about, or smaller than, the size of the laser spot. It has been found that when this is achieved, the results from the mass spectrometric analysis are quantitative. The area under the peaks in the resulting mass spectra are proportional to concentration (when normalized and corrected for background). Methods for preparing and using such chips are described in U.S. Pat. No. 6,024,925, co-pending U.S. application Ser. Nos. 08/786,988, 09/364,774, 09/371,150 and 09/297,575; see, also PCT Application No. PCT/US97/20195 (WO 98/20020). Chips and kits for performing these analyses are commercially available from SEQUENOM under the trademark MassARRAY™. MassARRAY™ relies on the fidelity of the enzymatic primer extension reactions combined with the miniaturized array and MALDI-TOF (Matrix-Assisted Laser Desorption Ionization-Time of Flight) mass spectrometry to deliver results rapidly. It accurately distinguishes single base changes in the size of DNA fragments relating to genetic variants without tags.

Multiplex methods allow for the simultaneous detection of more than one polymorphic region in a particular gene. This is the typical method for carrying out haplotype analysis of allelic variants of the AKAP10 gene.

Multiplexing can be achieved by several different methodologies. For example, several mutations can be simultaneously detected on one target sequence by employing corresponding detector (probe) molecules (e.g., oligonucleotides or oligonucleotide mimetics). The molecular weight differences between the detector oligonucleotides must be large enough so that simultaneous detection (multiplexing) is possible. This can be achieved either by the sequence itself (composition or length) or by the introduction of mass-modifying functionalities into the detector oligonucleotides (see below).

Mass modifying moieties can be attached, for instance, to either the 5′-end of the oligonucleotide, to the nucleobase (or bases), to the phosphate backbone, and to the 2′-position of the nucleoside (nucleosides) and/or to the terminal 3′-position. Examples of mass modifying moieties include, for example, a halogen, an azido, or of the type, XR, wherein X is a linking group and R is a mass-modifying functionality. The mass-modifying functionality can thus be used to introduce defined mass increments into the oligonucleotide molecule.

The mass-modifying functionality can be located at different positions within the nucleotide moiety (see, e.g., U.S. Pat. No. 5,547,835 and International PCT Application No. WO 94/21822). For example, the mass-modifying moiety, M, can be attached either to the nucleobase, (in case of the c⁷-deazanucleosides also to C-7), to the triphosphate group at the alpha phosphate or to the 2′-position of the sugar ring of the nucleoside triphosphate. Modifications introduced at the phosphodiester bond, such as with alpha-thio nucleoside triphosphates, have the advantage that these modifications do not interfere with accurate Watson-Crick base-pairing and additionally allow for the one-step post-synthetic site-specific modification of the complete nucleic acid molecule e.g., via alkylation reactions (see, e.g., Nakamaye et al. (1988) Nucl. Acids Res. 16:9947-59). Typical mass-modifying functionalities are boron-modified nucleic acids since they are better incorporated into nucleic acids by polymerases (see, e.g., Porter et al. (1995) Biochemistry 34:11963-11969; Hasan et al. (1996) Nucleic Acids Res. 24:2150-2157; Li et al. (1995) Nucl. Acids Res. 23:4495-4501).

Furthermore, the mass-modifying functionality can be added so as to affect chain termination, such as by attaching it to the 3′-position of the sugar ring in the nucleoside triphosphate. For those skilled in the art, it is clear that many combinations can be used in the methods provided herein. In the same way, those skilled in the art will recognize that chain-elongating nucleoside triphosphates can also be mass-modified in a similar fashion with numerous variations and combinations in functionality and attachment positions.

For example, without being bound to any particular theory, the mass-modification can be introduced for X in XR as well as using oligo-/polyethylene glycol derivatives for R. The mass-modifying increment (m) in this case is 44, i.e. five different mass-modified species can be generated by just changing m from 0 to 4 thus adding mass units of 45 (m=0), 89 (m=1), 133 (m=2), 177 (m=3) and 221 (m=4) to the nucleic acid molecule (e.g., detector oligonucleotide (D) or the nucleoside triphosphates, respectively). The oligo/polyethylene glycols can also be monoalkylated by a lower alkyl such as, but are not limited to, methyl, ethyl, propyl, isopropyl and t-butyl. Other chemistries can be used in the mass-modified compounds (see, e.g., those described in Oligonucleotides and Analogues, A Practical Approach, F. Eckstein, editor, IRL Press, Oxford, 1991).

In yet another embodiment, various mass-modifying functionalities, R, other than oligo/polyethylene glycols, can be selected and attached via appropriate linking chemistries, X. A simple mass-modification can be achieved by substituting H for halogens, such as F, Cl, Br and/or I, or pseudohalogens such as CN, SCN, NCS, or by using different alkyl, aryl or aralkyl moieties such as methyl, ethyl, propyl, isopropyl, t-butyl, hexyl, phenyl, substituted phenyl, benzyl, or functional groups such as CH₂F, CHF₂, CF₃, Si(CH₃)₃, Si(CH₃)₂(C₂H₅), Si(CH₃)(C₂H₅)₂, Si(C₂H₅)₃. Yet another mass-modification can be obtained by attaching homo- or heteropeptides through the nucleic acid molecule (e.g., detector (D)) or nucleoside triphosphates). One example, useful in generating mass-modified species with a mass increment of 57, is the attachment of oligoglycines (m) to nucleic acid molecules (r), e,g., mass-modifications of 74 (r=1, m=0), 131 (r=1, m=1), 188 (r=1, m=2), 245 (r=1, m=3) are achieved. Simple oligoamides also can be used, e.g., mass-modifications of 74 (r=1, m=0), 88 (r=2, m=0), 102 (r=3, m=0), 116(r=4, m=0), etc. are obtainable. Variations in additions to those set forth herein will be apparent to the skilled artisan.

Different mass-modified detector oligonucleotides can be used to simultaneously detect all possible variants/mutants simultaneously. Alternatively, all four base permutations at the site of a mutation can be detected by designing and positioning a detector oligonucleotide, so that it serves as a primer for a DNA/RNA polymerase with varying combinations of elongating and terminating nucleoside triphosphates. For example, mass modifications also can be incorporated during the amplification process.

A different multiplex detection format is one in which differentiation is accomplished by employing different specific capture sequences which are position-specifically immobilized on a flat surface (e.g., a ‘chip array’). If different target sequences T1-Tn are present, their target capture sites TCS1-TCSn will specifically interact with complementary immobilized capture sequences C1-Cn. Detection is achieved by employing appropriately mass differentiated detector oligonucleotides D1-Dn, which are mass modifying functionalities M1-Mn.

o. Other Methods

Additional methods of analyzing nucleic acids include amplification- based methods including polymerase chain reaction (PCR), ligase chain reaction (LCR), mini-PCR, rolling circle amplification, autocatalytic methods, such as those using QJ replicase, TAS, 3SR, and any other suitable method known to those of skill in the art.

Other methods for analysis and identification and detection of polymorphisms, include but are not limited to, allele specific probes, Southern analyses, and other such analyses.

2. Primers, Probes and Antisense Nucleic Acid Molecules

Primers refer to nucleic acids which are capable of specifically hybridizing to a nucleic acid sequence which is adjacent to a polymorphic region of interest or to a polymorphic region and are extended. A primer can be used alone in a detection method, or a primer can be used together with at least one other primer or probe in a detection method. Primers can also be used to amplify at least a portion of a nucleic acid. For amplifying at least a portion of a nucleic acid, a forward primer (i.e., 5′ primer) and a reverse primer (i.e., 3′ primer) will typically be used. Forward and reverse primers hybridize to complementary stands of a double stranded nucleic acid, such that upon extension from each primer, a double stranded nucleic acid is amplified.

Probes refer to nucleic acids which hybridize to the region of interest and which are not further extended. For example, a probe is a nucleic acid which hybridizes adjacent to or at a polymorphic region of an AKAP gene and which by hybridization or absence of hybridization to the DNA of a subject will be indicative of the identity of the allelic variant of the polymorphic region of the gene. Typical probes have a number of nucleotides sufficient to allow specific hybridization to the target nucleotide sequence. Where the target nucleotide sequence is present in a large fragment of DNA, such as a genomic DNA fragment of several tens or hundreds of kilobases, the size of a probe can have to be longer to provide sufficiently specific hybridization, as compared to a probe which is used to detect a target sequence which is present in a shorter fragment of DNA. For example, in some diagnostic methods, a portion of an AKAP gene can first be amplified and thus isolated from the rest of the chromosomal DNA and then hybridized to a probe. In such a situation, a shorter probe will likely provide sufficient specificity of hybridization. For example, a probe having a nucleotide sequence of about 10 nucleotides can be sufficient.

Primers and probes (RNA, DNA (single-stranded or double-stranded), PNA and their analogs) described herein can be labeled with any detectable reporter or signal moiety including, but not limited to radioisotopes, enzymes, antigens, antibodies, spectrophotometric reagents, chemiluminescent reagents, fluorescent and any other light producing chemicals. Additionally, these probes can be modified without changing the substance of their purpose by terminal addition of nucleotides designed to incorporate restriction sites or other useful sequences, proteins, signal generating ligands such as acridinium esters, and/or paramagnetic particles.

These probes can also be modified by the addition of a capture moiety (including, but not limited to para-magnetic particles, biotin, fluorescein, dioxigenin, antigens, antibodies) or attached to the walls of microtiter trays to assist in the solid phase capture and purification of these probes and any DNA or RNA hybridized to these probes. Fluorescein can be used as a signal moiety as well as a capture moiety, the latter by interacting with an anti-fluorescein antibody.

Any probe, primer or antisense molecule can be prepared according to methods well known in the art and described, e.g., in Sambrook, J. Fritsch, E. F., and Maniatis, T. (1989(Molecular Cloning: A Laboratory Manual, Cold Spring Harbor Laboratory Press, Cold Spring Harbor, N.Y. For example, discrete fragments of the DNA can be prepared and cloned using restriction enzymes. Alternatively, probes and primers can be prepared using the Polymerase Chain Reaction (PCR) using primers having an appropriate sequence.

Oligonucleotides can be synthesized by standard methods known in the art, e.g. by use of an automated DNA synthesizer (such as are commercially available from Biosearch (Novato, Calif.); Applied Biosystems (Foster City, Calif.) and other methods). As examples, phosphorothioate oligonucleotides can be synthesized by the method of Stein et al. (1988, Nucl. Acids Res. 16:3209), methylphosphonate oligonucleotides, for example, can be prepared by use of controlled pore glass polymer supports (Sarin et al., 1988, Proc. Natl. Acad. Sci. U.S.A. 85:7448-7451).

Probes and primers used in the methods of detecting allelic variants in human AKAP10 genes are of sufficient length to specifically hybridize to portions of AKAP10 gene at polymorphic sites. Typically such lengths depend upon the complexity of the source organism genome. For humans such lengths are at least 14-16 nucleotides, and typically can be 20, 30, 50, 100 or more nucleotides.

The methods of detecting polymorphisms in human AKAP10 genes provided herein, probes and primers include the following:

(1) at least 14 or 16 contiguous nucleotides of the AKAP10 allele or complement thereof, wherein nucleic acid molecule includes at least 5 contiguous nucleotides from nucleotide 2069 to nucleotide 2077 of SEQ. ID. NO: 3;

(2) at least 14 or 16 contiguous nucleotides of the AKAP10 allele or complement thereof, wherein the nucleic acid includes the nucleotide at position 2073 of SEQ ID No. 1 replaced with G, C or T.

(3) at least 14 or 16 contiguous nucleotides of the AKAP10 allele or complement thereof, wherein the nucleic acid molecule includes at least 5 contiguous nucleotides from nucleotide 129556 to nucleotide 129604 of SEQ. ID. NO: 14;

(4) at least 14 or 16 contiguous nucleotides of the AKAP10 allele or complement thereof, wherein the nucleic acid includes the nucleotide at position 129600 of SEQ ID No. 17 replaced with A, C or T;

(5) at least 14 or 16 contiguous nucleotides of the AKAP10 allele or complement thereof, wherein the nucleic acid molecule includes at least 5 contiguous nucleotides from nucleotide 83583 to nucleotide 83591 of SEQ. ID. NO: 13;

(6) at least 14 or 1 6 contiguous nucleotides of the AKAP10 allele or complement thereof, wherein the nucleic acid includes the nucleotide at position 83587 of SEQ ID No. 17 replaced with G, A or T;

(7) at least 14 or 16 contiguous nucleotides of the AKAP10 allele or complement thereof, wherein the nucleic acid molecule includes at least 5 contiguous nucleotides from nucleotide 156,273 to nucleotide 156281 of SEQ. ID. NO: 18;

(8) at least 14 or 16 contiguous nucleotides of the AKAP10 allele or complement thereof, wherein the nucleic acid includes the nucleotide at position 156277 of SEQ ID No. 17 replaced with C, A or G;

With respect to each of the above described probes and primers, they have fewer nucleotides than the sequence of nucleotides 138 to 2126 of SEQ. ID. NO: 1 or fewer nucleotides than the sequence of nucleotides 83,580 to 156,577 of SEQ ID NO: 17.

Antisense compounds can be conveniently and routinely made through the well-known technique of solid phase synthesis. Equipment for such synthesis is sold by several vendors including, for example, Applied Biosystems (Foster City, Calif.). Any other means for such synthesis known in the art can additionally or alternatively be employed. It is well known to use similar techniques to prepare oligonucleotides such as the phosphorothioates and alkylated derivatives.

Antisense compounds are typically 8 to 30 nucleotides in length complementary to a targeted to a nucleic acid molecule and modulates its expression. The targeted nucleic acid molecule represents the coding strand. For example, for the AKAP10-5 alleleic variant an antisense compound is an antisense oligonucleotide which comprises the complement of at least an 8 nucleotide segment of SEQ ID NO: 3 including the nucleotide at position 2073 of SEQ ID NO: 3.

An antisense compound can contain at least one modified nucleotide which can confer nuclease resistance or increase the binding of the antisense compound with the target nucleotide. The antisense compound can containing at least one internucleoside linkage wherein the modified internucleoside linkage of the antisense oligonucleotide can be a phosphorothioate linkage, a morpholino linkage or a peptide-nucleic acid linkage.

Typical modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos.: 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

An antisense compound can contain at least one least one modified sugar moiety wherein the modified sugar moiety of the antisense oligonucleotide is a 2′-O-methoxyethyl sugar moiety or a 2′-dimethylaminooxyethoxy sugar moiety.

Modified oligonucleotides can also contain one or more substituted sugar moieties. Typical oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary oligonucleotides contain are O[(CH₂)ₙO]ₘCH₃, O(CH₂)ₙOCH₃, O(CH₂)ₙNH₂, O(CH₂)ₙCH₃, O(CH₂)ₙNH₂, and O(CH₂)ₙON[(CH₂)ₙCH₃)]₂, where n and m are from 1 to about 10. Other oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A typical modification includes an alkoxyalkoxy group, 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504). Another exemplary modification includes 2′-dimethylaminooxyethoxy, i.e., a O(CH₂)₂ON(CH₃)₂ group, also known as 2′-DMAOE.

Other modifications include 2′-methoxy (2′-O—CH₃), 2′-aminopropoxy (2′-OCH₂CH₂CH₂NH₂) and 2′-fluoro (2′-F). Similar modifications can also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides can also have sugar mimetics such as cyclobutyl moieties in place of the pentofuranosyl sugar. Representative United States patents that teach the reparation of such modified sugar structures include, but are not limited to, U.S. Pat. Nos.: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,0531 5,639,873; 5,646,265; 5,658,873; 5,670,633; and 5,700,920, each of which is herein incorporated by reference.

An antisense compound can contain at least one modified nucleobase. Oligonucleotides can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Crooke, S. T., and Lebleu, B. eds., Antisense Research and Applications, CRC Press, Boca Raton, 1993, pp. 289-302. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyl-adenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are typical base substitutions, even more particularly when combined with 2′-O-methoxyethyl sugar modifications.

The antisense compound can be a chimeric oligonucleotide. Chimeric antisense compounds can be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos.: 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

E. Measuring and Electrocardiogram

Devices that can be used to measure an electrocardiogram are referred to as electrocardiographs. A variety of electrocardiographs are well known in the art and include, for example, those disclosed is U.S. Pat. Nos. 4,377,813, 4,483,346, 4,98,479, 4,840,183, 4,974599. Any known electrocardiograph can be used in the methods provided herein. Methods of using electrocardiographs to determine P-R interval and Q-T interval also are well known in the art, and any such method can be used in the methods provided herein.

F. Association of AKAP10 Allelic Variants with Morbidity or Increased Mortality

Polymorphisms of the genome can lead to altered gene function, protein function or mRNA instability. To identify those polymorphisms that have clinical relevance is the goal of a world-wide scientific effort. Discovery of such polymorphisms will have a fundamental impact on the identification and development of diagnostics and drug discovery. The strategy to identify valuable polymorphisms is cumbersome and dependent upon the availability of many large patient and control cohorts to show disease association. Furthermore, genes, and their associated polymorphisms, that cause a general risk of the population to suffer from any disease will escape these case/control studies entirely.

A morbidity susceptibility gene could be a gene that is expressed in many different cell types or tissues (housekeeping gene) and its altered function can facilitate the expression of a clinical phenotype caused by a disease-specific susceptibility gene that is involved in a pathway specific for this disorder. In other words, morbidity susceptibility genes might predispose people to develop a distinct disease according to their genetic make-up for this disease. Candidates for these genes can involve basic cellular processes such as: transcription, translation, heat-shock proteins, protein trafficking, DNA repair, assembly systems for subcellular structures (e.g., mitochondria, peroxysomes and other cellular microbodies), receptor signaling cascades, immunology, etc. Those pathways control the quality of life at the cellular level as well as for the entire organism. Mutations/polymorphisms located in genes encoding proteins for those pathways can reduce the fitness of cells and make the organism more susceptible to express the clinical phenotype caused by the action of a disease-specific susceptibility gene. Therefore, these morbidity susceptibility genes can be potentially involved in a whole variety of different complex diseases if not in all.

An example of possible candidate morbidity susceptibility genes are mutants of the A kinase anchoring protein (AKAP) genes. Protein phosphorylation is an important mechanism for enzyme regulation and signal transduction in eukaryotic cells. cAMP dependent protein kinase (PKA) mediates a variety of hormonal and neurotransmitter responses by phosphorylating a wide variety of substrates including enzymes, membrane receptors, ion channels and transcription factors. AKAPs direct the subcellular localization of cAMP-dependent protein kinase by binding to its regulatory subunits and therefore plays a role in G-protein mediated receptor-signaling pathways. (Huang et al. Proc. Natl. Acad. Sci., USA 94:11184, 1997). AKAPs have a PKA binding region located in their COOH-terminal portion.

Polymorphic AKAP genes, such as those provided herein, serve as markers for detecting predisposition to disease and various conditions. Also, the AKAP alleles and gene products, especially the AKAP10-5 gene product should be suitable pharmaceutical targets and gene therapy targets.

In a further allele, designated AKAP10-7 contains a single nucleotide polymorphism (SNP), a G-to-A transition, at nucleotide position 129,600 of the human chromosome 17 sequence (also referred to herein as SNP “In10”). This SNP is located four bases 3′ to the exon 10/intron 10 boundary of AKAP10 gene. Another identified SNP, AKAP10-1 is an allelic variant with a T to C transversion at nucleotide position 156,277 of the AKAP10 genomic clone which is located in the 3′ untranslated region of the gene (also referred to herein as SNP “3′ UTR”).

Utilizing a healthy patient database, the frequency of occurrence of two allelic variants of the AKAP10 gene, AKAP10-5 and AKAP10-1, in such a population were found to decrease with age. The AKAP10-5 and AKAP10-1 alleles are useful markers for predicting susceptibility to morbidity and/or increased or early mortality. The methods provided herein can be used for predicting susceptibility to morbidity, increased or early mortality, or morbidity and increased mortality, by detecting the presence of the various AKAP10 allelic variants known in the art or dislcosed herein, individually, or in combination with other AKAP10 allelic variants, in an organism, particularly an animal and particularly a human. AKAP10-5 and other allelic variants of the AKAP10 gene known in the art or dislcosed herein are potential functional variants of a morbidity susceptibility gene and/or of a gene involved in increased mortality and/or a gene related to an alteration in signal transduction and associated disorders and thus is useful for screening for potential therapeutics.

G. Effect of Allelic Variants

The effect of an allelic variant on a AKAP10 gene expression (amount of mRNA, mRNA stability) and AKAP protein (amount, stability, intracellular localization, activity) can be determined according to methods known in the art. Allelic variants of AKAP genes can be assayed individually or in combination.

In general, any method known to those skilled in the art of determining the presence or absence of a specific messenger RNA transcript or a specific translated protein can be used to presence of absence of a polymorphic protein or a polymorphism in the genetic sequence.

1. RNA Analysis

a. Northern Blot Detection of RNA

The northern blot technique is used to identify a RNA fragment of a specific size from a complex population of RNA using gel electrophoresis and nucleic acid hybridization. Northern blotting is a well-known technique in the art. Northern blot analysis is commonly used to detect specific RNA transcripts expressed in a variety of biological samples and have been described in Sambrook, J. et al. (Molecular Cloning, 3^{rd} Edition, Cold Spring Harbor Press).

Briefly, total RNA is isolated from any biological sample by the method of Chomczynski and Sacchi (Anal. Biochem. (1987) 162, 156-159). Poly-adenylated mRNA is purified from total RNA using mini-oligo (dT) cellulose spin column kit with methods as outlined by the suppliers (Invitrogen, Carlsbad Calif.). Denatured RNA is electrophoresed through a denaturing 1.5% agarose gel and transferred onto a nitrocellulose or nylon based matrix. The mRNAs are detected by hybridization of a radiolabeled or biotinylated oligonucleotide probe specific to the polymorphic regions as disclosed herein.

b. Dot Blot/Slot Blot

Specific RNA transcripts can be detected using dot and slot blot assays to evaluate the presence of a specific nucleic acid sequence in a complex mix of nucleic acids. Specific RNA transcripts can be detected by adding the RNA mixture to a prepared nitrocellulose or nylon membrane. RNA is detected by the hybridization of a radiolabeled or biotinylated oligonucleotide probe complementary to the AKAP sequences as disclosed herein.

c. RT-PCR

The RT-PCR reaction can be performed, as described by K.-Q. Hu et al., Virology 181:721-726 (1991), as follows: the extracted mRNA is transcribed in a reaction mixture 1 micromolar antisense primer, and 25 U AMV (avian myeloblastosis virus) or MMLV (Moloney murine leukemia virus) reverse transcriptase. Reverse transcription is performed and the cDNA is amplified in a PCR reaction volume with Taq polymerase. Optimal conditions for cDNA synthesis and thermal cycling can be readily determined by those skilled in the art.

2. Protein and Polypeptide Detection

a. Expression of Protein in a Cell Line

Using the disclosed nucleic acids AKAP10 proteins can be expressed in a recombinantly engineered cell such as bacteria, yeast, insect, mammalian, or plant cells. Those of skill in the art are knowledgeable in the numerous expression systems available for expression of a nucleic acid encoding proteins such as polymorphic human AKAP10 proteins.

b. Expression of AKAP Protein

The isolated nucleic acid encoding a full-length polymorphic human AKAP10 protein, or a portion thereof, such as a fragment containing the site of the polymorphism, can be introduced into a vector for transfer into host cells. Fragments of the polymorphic human AKAP10 proteins can be produced by those skilled in the art, without undue experimentation, by eliminating portions of the coding sequence from the isolated nucleic acids encoding the full-length proteins.

Expression vectors are used expression of the protein in the host cell is desired. An expression vector includes vectors capable of expressing nucleic acids that are operatively linked with regulatory sequences, such as promoter regions, that are capable of effecting expression of such nucleic acids. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome. Such plasmids for expression of polymorphic human AKAP10-encoding nucleic acids in eukaryotic host cells, particularly mammalian cells, include cytomegalovirus (CMV) promoter-containing vectors, such as pCMV5, the pSV2dhfr expression vectors, which contain the SV40 early promoter, mouse dhfr gene, SV40 polyadenylation and splice sites and sequences necessary for maintaining the vector in bacteria, and MMTV promoter-based vectors.

The nucleic acids encoding polymorphic human AKAP10 proteins, and vectors and cells containing the nucleic acids as provided herein permit production of the polymorphic proteins, as well as antibodies to the proteins. This provides a means to prepare synthetic or recombinant polymorphic human AKAP10 proteins and fragments thereof that are substantially free of contamination from other AKAPs and proteins in general, the presence of which can interfere with analysis of the polymorphic proteins. In addition, the polymorphic proteins can be expressed in combination with selected other proteins that AKAP10 can associate with in cells. The ability to selectively express the polymorphic AKAP10 proteins alone or in combination with other selected proteins makes it possible to observe the functioning of the recombinant polymorphic proteins within the environment of a cell. The expression of isolated nucleic acids encoding an AKAP protein will typically be achieved by operably linking, for example, the DNA or cDNA to a promoter (which is either constitutive or regulatable), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein. To obtain high level expression of a cloned gene, it is desirable to construct expression vectors which contain, a strong promoter to direct transcription, a ribosome binding site for translational initiation, and a transcription/translation terminator. One of skill in the art would recognize that modifications can be made to an AKAP10 protein without diminishing its biological activity. Some modifications can be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located purification sequences. Restriction sites or termination codons can also be introduced. There are expression vectors that specifically allow the expression of functional proteins. One such vector, Plasmid 577, described in U.S. Pat. No. 6,020,122 and incorporated herein by reference, has been constructed for the expression of secreted antigens in a permanent cell line. This plasmid contains the following DNA segments: (a) a fragment of pBR322 containing bacterial beta-lactamase and origin of DNA replication; (b) a cassette directing expression of a neomycin resistance gene under control of HSV-1 thymidine kinase promoter and poly-A addition signals; (c) a cassette directing expression of a dihydrofolate reductase gene under the control of a SV-40 promoter and poly-A addition signals; (d) cassette directing expression of a rabbit immunoglobulin heavy chain signal sequence fused to a modified hepatitis C virus (HCV) E2 protein under the control of the Simian Virus 40 T-Ag promoter and transcription enhancer, the hepatitis B virus surface antigen (HBsAg) enhancer I followed by a fragment of Herpes Simplex Virus-1 (HSV-1) genome providing poly-A addition signals; and (e) a fragment of Simian Virus 40 genome late region of no function in this plasmid. All of the segments of the vector were assembled by standard methods known to those skilled in the art of molecular biology. Plasmids for the expression of secreted AKAP proteins can be constructed by replacing the hepatitis C virus E2 protein coding sequence in plasmid 577 with a AKAP sequence of SEQ ID NO: 3 or a fragment thereof. The resulting plasmid is transfected into CHO/dhfr-cells (DXB-111) (Uriacio, et al., PNAS 77, 4451-4466; 1980); these cells are available from the A.T.C.C., 12301 Parklawn Drive, Rockville, Md. 20852, under Accession No. CRL 9096), using the cationic liposome-mediated procedure (P. L. Feigner et al., PNAS 84:7413-7417 (1987). Proteins are secreted into the cell culture media.

Incorporation of cloned DNA into a suitable expression vector, transfection of cells with a plasmid vector or a combination of plasmid vectors, each encoding one or more distinct proteins or with linear DNA, and selection of transfected cells are well known in the art (see, e.g., Sambrook et al. (1989) Molecular Cloning: A Laboratory Manual, Second Edition, Cold Spring Harbor Laboratory Press). Heterologous nucleic acid can be introduced into host cells by any method known to those of skill in the art, such as transfection with a vector encoding the heterologous nucleic acid by CaPO₄ precipitation (see, e.g., Wigler et al. (1979) Proc. Natl. Acad. Sci. USA 76:1373-1376) or lipofectamine (GIBCO BRL #18324-012). Recombinant cells can then be cultured under conditions whereby the polymorphic human AKAP10 protein encoded by the nucleic acid is expressed. Suitable host cells include mammalian cells (e.g., HEK293, including but are not limited to, those described in U.S. Pat. No. 5,024,939 to Gorman (see, also, Stillman et al. (1985) Mol. Cell. Biol. 5:2051-2060); also, HEK293 cells available from ATCC under accession #CRL 1573), CHO, COS, BHKBI and Ltk⁻ cells, mouse monocyte macrophage P388D1 and J774A-1 cells (available from ATCC, Rockville, Md.) and others known to those of skill in this art), yeast cells, including, but are not limited to, Pichia pastoris, Saccharomyces cerevisiae, Candida tropicalis, Hansenula polymorpha, human cells and bacterial cells, including, but are not limited to, Escherichia coli. Xenopus oöcytes can also be used for expression of in vitro RNA transcripts of the DNA.

Heterologous nucleic acid can be stably incorporated into cells or can be transiently expressed using methods known in the art. Stably transfected mammalian cells can be prepared by transfecting cells with an expression vector having a selectable marker gene (such as, for example, the gene for thymidine kinase, dihydrofolate reductase, neomycin resistance, and the like), and growing the transfected cells under conditions selective for cells expressing the marker gene. To prepare transient transfectants, mammalian cells are transfected with a reporter gene (such as the E. coli β-galactosidase gene) to monitor transfection efficiency. Selectable marker genes are not included in the transient transfections because the transfectants are typically not grown under selective conditions, and are usually analyzed within a few days after transfection.

Heterologous nucleic acid can be maintained in the cell as an episomal element or can-be integrated into chromosomal DNA of the cell. The resulting recombinant cells can then be cultured or subcultured (or passaged, in the case of mammalian cells) from such a culture or a subculture thereof. Methods for transfection, injection and culturing recombinant cells are known to the skilled artisan. Similarly, the polymorphic human AKAP10 proteins or fragments thereof can be purified using protein purification methods known to those of skill in the art. For example, antibodies or other ligands that specifically bind to the proteins can be used for affinity purification and immunoprecipitation of the proteins.

b. Protein Purification

The AKAP10 proteins can be purified by standard techniques well known to those of skill in the art. Recombinantly produced proteins can be directly expressed or expressed as a fusion protein. The recombinant protein is purified by a combination of cell lysis (e.g., sonication, French press) and affinity chromatography. The proteins, recombinant or synthetic, can be purified to substantial purity by standard techniques well known in the art, including detergent solubilization, selective precipitation with such substances as ammonium sulfate, column chromatography, immunopurification methods, and others. (See, for example, R. Scopes, Protein Purification: Principles and Practice, Springer-Verlag: New York (1982); Deutscher, Guide to Protein Purification, Academic Press (1990)). For example, antibodies can be raised to the proteins as described herein. Purification from E. coli can be achieved following procedures described in U.S. Pat. No. 4,511,503. The protein can then be isolated from cells expressing the protein and further purified by standard protein chemistry techniques as described herein. Detection of the expressed protein is achieved by methods known in the art and include, for example, radioimmunoassays, Western blotting techniques or immunoprecipitation.

3. Immunodetection of the AKAP10 Protein

Generally, the AKAP proteins, when presented as an immunogen, should elicit production of a specifically reactive antibody. Immunoassays for determining binding are well known to those of skill in the art, as are methods of making and assaying for antibody binding specificity/affinity. Exemplary immunoassay formats include ELISA, competitive immunoassays, radioimmunoassays, Western blots, indirect immunofluorescent assays, in vivo expression or immunization protocols with purified protein preparations. In general, the detection of immunocomplex formation is well known in the art and can be achieved by methods generally based upon the detection of a label or marker, such as any of the radioactive, fluorescent, biological or enzymatic tags. Labels are well known to those skilled in the art (see U.S. Pat. Nos. 3,817,837; 3,850,752; 3,939,350; 3,996,345; 4,277,437; 4,275,149 and 4,366,241, each incorporated herein by reference). Of course, one can find additional advantages through the use of a secondary binding ligand such as a second antibody or a biotin/avidin ligand binding arrangement, as is known in the art.

a. Production of Polyclonal Antisera Against AKAP

Antibodies can be raised to AKAP proteins, including individual, allelic, strain, or species variants, and fragments thereof, both in their naturally occurring (full-length) forms and in recombinant forms. Additionally, antibodies are raised to these proteins in either their native configurations or in non-native configurations. Anti-idiotypic antibodies can also be generated. A variety of analytic methods are available to generate a hydrophilicity profile of proteins. Such methods can be used to guide the artisan in the selection of peptides for use in the generation or selection of antibodies which are specifically reactive, under immunogenic conditions. See, e.g., J. Janin, Nature, 277 (1979) 491-492; Wolfenden, et al., Biochemistry 20(1981) 849-855; Kyte and Doolite, J. Mol. Biol. 157 (1982) 105-132; Rose, et al., Science 229 (1985) 834-838.

A number of immunogens can be used to produce antibodies specifically reactive with AKAP proteins. Isolated recombinant, synthetic, or native polypeptides are typical immunogens (antigen) for the production of monoclonal or polyclonal antibodies. Polypeptides are typically denatured, and optionally reduced, prior to formation of antibodies for screening expression libraries or other assays in which a putative AKAP protein is expressed or denatured in a non-native secondary, tertiary, or quartenary structure.

The AKAP protein (SEQ ID NO: 4, or a portion thereof) is injected into an animal capable of producing antibodies. Either monoclonal or polyclonal antibodies can be generated for subsequent use in immunoassays to measure the presence and quantity of the protein. Methods of producing polyclonal antibodies are known to those of skill in the art. In brief, an immunogen (antigen), typically a purified protein, a protein coupled to an appropriate carrier (e.g., GST, keyhole limpet hemanocyanin, etc.), or a protein incorporated into an immunization vector such as a recombinant vaccinia virus (see, U.S. Pat. No. 4,722,848) is mixed with an adjuvant and animals are immunized with the mixture. The animal's immune response to the immunogen preparation is monitored by taking test bleeds and determining the titer of reactivity to the protein of interest. When appropriately high titers of antibody to the immunogen are obtained, blood is collected from the animal and antisera are prepared. Further fractionation of the antisera to enrich for antibodies reactive to the protein is performed where desired (See, e.g., Coligan, Current Protocols in Immunology, Wiley/Greene, NY (1991); and Harlow and Lane, Antibodies: A Laboratory Manual, Cold Spring Harbor Press, NY (1989)).

b. Western Blotting of Tissue Samples for the AKAP Protein

Biological samples are homogenized in SDS-PAGE sample buffer (50 mM Tris-HCl, pH 6.8, 100 mM dithiothreitol, 2% SDS, 0.1% bromophenol blue, 10% glycerol), heated at 100 degrees Celsius for 10 min and run on a 14% SDS-PAGE with a 25 mM Tris-HCl, pH 8.3, 250 mM Glycine, 0.1% SDS running buffer. The proteins are electrophoretically transferred to nitrocellulose in a transfer buffer containing 39 mM glycine, 48 mM Tris-HCl, pH 8.3, 0.037% SDS, 20% methanol. The nitrocellulose is dried at room temperature for 60 min and then blocked with a PBS solution containing either bovine serum albumin or 5% nonfat dried milk for 2 hours at 4 degrees Celsius.

The filter is placed in a heat-sealable plastic bag containing a solution of 5% nonfat dried milk in PBS with a 1:100 to 1:2000 dilution of affinity purified anti-AKAP peptide antibodies, incubated at 4 degrees Celsius for 2 hours, followed by three 10 min washes in PBS. An alkaline phosphatase conjugated secondary antibody (i.e., anti-mouse/rabbit IgG), is added at a 1:200 to 1:2000 dilution to the filter in a 150 mM NaCl, 50 mM Tris-HCl, pH 7.5 buffer and incubated for 1 h at room temperature.

The bands are visualized upon the addition and development of a chromogenic substrate such as 5-bromo-4-chloro-3-indolyl phosphate/nitro blue tetrazolium (BCIP/NBT). The filter is incubated in the solution at room temperature until the bands develop to the desired intensity. Molecular mass determination is made based upon the mobility of pre-stained molecular weight standards (Rainbow markers, Amersham, Arlington Heights, Ill.).

c. Microparticle Enzyme Immunoassay (MEIA)

AKAP10 proteins and peptides are detected using a standard commercialized antigen competition EIA assay or polyclonal antibody sandwich EIA assay on the IMx.RTM Analyzer (Abbott Laboratories, Abbott Park, Ill.). Samples containing the AKAP10 protein are incubated in the presence of anti-AKAP10 coated microparticles. The microparticles are washed and secondary polyclonal anti-AKAP10 antibodies conjugated with detectable entities (i.e., alkaline phosphatase) are added and incubated with the microparticles. The microparticles are washed and the bound antibody/antigen/antibody complexes are detected by adding a substrate (i.e. 4-methyl umbelliferyl phosphate) (MUP) that will react with the secondary conjugated antibody to generate a detectable signal.

d. Immunocytochemistry

Intracellular localization of the AKAP10 protein can be determined by a variety of in situ hybridization techniques. In one method cells are fixed with fixed in 4% paraformaldehyde in 0.1 M phosphate buffered saline (PBS; pH 7.4) for 5 min., rinsed in PBS for 2 min., dilapidated and dehydrated in an ethanol series (50, 70 and 95%) (5 min. each and stored in 95% ethanol at 4 degrees Celsius).

The cells are stained with the primary anti-AKAP10 antibody and a mixture of secondary antibodies used for detection. Laser-scanning confocal microscopy is performed to localize the AKAP10 protein.

4. Binding Assays

Assays to measure the interaction between AKAP10 and the regulatory subunits RI and/or RII of the Protein Kinase A holoenzyme include immobilized binding assays, solution binding assays and the like. In some instances, it can be desirable to monitor binding between AKAP10 and PKA. In other instances, it can be desirable to specifically monitor the binding between AKAP10 and a cellular component (other than PKA) to which it binds. Assays can be performed in a variety of formats, including cell-based assays, such as di-hybrid screening or complementation assays as described in U.S. Pat. No. 5,283,173 and Patent Cooperation Treaty (PCT) Publication No. WO 91/16457, respectively. Assays of this type are particularly useful for assessing intracellular efficacy of test compounds. Non-cell-based assays include scintillation proximity assays, cAMP competition assays, ELISA assays, radioimmunoassays, chemiluminescent assays, and the like. Such assay procedures are well known in the art and generally described, e.g., in Boudet et al., J. Immunol. Meth., 142:73-82 (1991); Ngai et al., J. Immunol. Meth., 158:267-276 (1993); Pruslin et al., J. Immunol. Meth., 137:27-35 (1991); Udenfriend et al., Proc. Natl. Acad. Sci. USA, 82:8672-8676 (1985); Udenfriend et al., Anal. Biochem., 161:494-500 (1987); Bosworth and Towers, Nature, 341:167-168 (1989); Gilman, Proc. Natl. Acad. Sci. USA, 67:305-312 (1970); and U.S. Pat. No. 4,568,649.

a. In Vitro Binding Assay

Huang et al. Proc. Natl. Acad. Sci. USA, 272:8057-8064 (1997); Protein preparations containing AKAP10 are incubated with glutathione resin in PBS for 2 hours at 4 degrees Celsius with 0.1% Triton X-100, 1 mM phenylmethylsulfonyl fluoride, 1 mM EDTA, 5 mM benzamidine, and 5 mM B-mercapthoethanol and washed extensively with the same buffer. 200 micrograms of PKA regulatory subunit RII and/or RI were added to the resin and incubated at 4 degrees Celsius. Proteins associated with the AKAP10 are eluted and analyzed by Laemmli electrophoresis. The proteins were visualized by Coomassie Staining. PKA proteins can be radiolabeled or labeled with a flurophore to allow detection.

b. PKA Phosphorylation of Protein Substrate

Cyclic AMP-dependent protein kinase (PKA) catalyzes the transfer of gamma phosphate from adenosine triphosphate (ATP) to a serine or threonine residue in a protein substrate. A short synthetic peptide (Leucine-Arg-Arg-Alanine-Serine-Leucine-Glycine or LRRASLG) is used as a substrate to assay the specific type of PKA activity as described in Pearson et. al., Methods of Enzymology 200, 62-81 (1991).

The PKA assay is typically carried out in a reaction of the enzyme with a peptide substrate and gamma 32P-ATP followed by separation of the 32P-peptide product from the unreacted gamma 32P-ATP on a phosphocellulose membrane. This method requires at least one basic amino acid residue in the peptide substrate. The peptide substrate can be tagged with a biotin group so that the biotinylated 32P-peptide product consistently binds to a streptavidin membrane in a manner independent of the peptide sequence as described in Goueli et al Analytical Biochemistry 225, 10-17, (1995). The separation of the 32P-peptide product from the free gamma 32P-ATP using affinity binding and ultrafiltration separation to analyze a mixture sample as described in U.S. Pat. No. 5,869,275.

If the mutation is located in an intron, the effect of the mutation can be determined, e.g., by producing transgenic animals in which the allelic variant has been introduced and in which the wild-type gene or predominant allele can have been knocked out. Comparison of the level of expression of the protein in the mice transgenic for the allelic variant with mice transgenic for the predominant allele will reveal whether the mutation results in increased or decreased synthesis of the associated protein and/or aberrant tissue distribution or intracellular localization of the associated protein. Such analysis could also be performed in cultured cells, in which the human variant allele gene is introduced and, e.g., replaces the endogenous gene in the cell. For mutant AKAP proteins binding to signaling enzymes such as PKA is also examined. Thus, depending on the effect of the alteration a specific treatment can be administered to a subject having such a mutation. Accordingly, if the mutation results in decreased production of AKAP protein, the subject can be treated by administration of a compound which increases synthesis, such as by increasing AKAP gene expression, and wherein the compound acts at a regulatory element different from the one which is mutated. Alternatively, if the mutation results in increased AKAP protein, the subject can be treated by administration of a compound which reduces protein production, e.g., by reducing AKAP gene expression or a compound which inhibits or reduces the activity of AKAP protein.

H. Diagnostic and Prognostic Assays

Typically, an individual allelic variant that associates with morbidity and/or mortality and/or an alteration in signal transduction will not be used in isolation as a prognosticator. An allelic variant typically will be one of a plurality of indicators that are used. The other indicators can be the manifestation of other risk factors for morbidity and/or mortality and other evidence of altered signal transduction.

Useful combinations of allelic variants of the AKAP10 gene can be determined. Variants can be assayed individually or assayed simultaneously using multiplexing methods as described above or any other labelling method that allows different variants to be identified. In particular, variants of the AKAP10 gene can be assayed using kits (see below) or any of a variety microarrays known to those in the art. For example, oligonucleotide probes comprising the polymorphic regions surrounding any polymorphism in the AKAP10 gene can be designed and fabricated using methods such as those described in U.S. Pat. Nos. 5,492,806; 5,525,464; 5,695,940; 6,018,041; 6,025,136; WO 98/30883; WO 98/56954; WO99/09218; WO 00/58516; WO 00/58519, or references cited therein.

I. Databases

Use of databases containing sets of parameters associated with subjects in populations selected on the basis of apparent good health, not manifesting detectable disease (i.e., an unbiased population not selected for any disease state), allows for identification of such morbidity susceptibility genes (see, U.S. Provisional Application Ser. No. 60/159,176 filed Oct. 13, 1999, U.S. Provisional Application Ser. No. 60/217,658 filed on Jul. 10, 2000 and U.S. application Ser. No. 09/687,483 filed Oct. 13, 2000).

For example, in a method for determining susceptibility to morbidity, increased or early mortality, or morbidity and increased or early mortality in a human being, provided herein, exemplary steps include detecting the presence or absence of an allele of the human AKAP10 containing other than an A at position 2073 of the coding sequence of the AKAP10 gene; wherein the presence of an allele containing other than an A at position 2073 is indicative of increased susceptibility to morbidity, increased or early mortality, or morbidity and increased or early mortality as compared to the susceptibility of a human being who does not comprise an allele containing other than an A at position 2073 of the AKAP10 gene coding sequence.

As noted above, using a healthy patient database (see, U.S. Provisional Application Ser. No. 60/159,176, U.S. Provisional Application Ser. No. 60/217,658 and U.S. application Ser. No. 09/687,483 filed Oct. 13, 2000), the frequency of occurrence of the AKAP10-5 SNP in such a population was found to decrease with age, thus making the allele a potential morbidity susceptibility gene, a gene associated with increased mortality or both. Using the healthy database, it was found that the homozygote GG genotype drops in the elderly population (over>60 years), by a statistically significant amount, p=0.02.

J. Isolation of Polymorphic AKAP10 Gene Sequences

Exemplary nucleic acid sequences encoding polymorphic human AKAP10 proteins are represented by nucleotides which encode the amino acid sequence as set forth in SEQ. ID. NO: 3. Such polymorphic nucleotide sequences can encode variant amino acid sequences, such as the sequence set forth in SEQ. ID. NO: 4 in which amino acid 646 has been replaced with a valine; other amino acid sequence variants at amino acid 646 include leucine or phenylalanine.

Other exemplary nucleic acid sequences represent allelic variants of the AKAP10 gene which are not located in protein coding regions. Such as set forth in nucleotide position 83,580 to position 156,577 of SEQ ID NO: 13, 14 and 18.

Nucleic acid encoding polymorphic human AKAP10 proteins and genes provided herein can be isolated by screening suitable human cDNA or human genomic libraries under suitable hybridization conditions with nucleic acids such as those provided in SEQ. ID. NOS: 1, 3, 13, 14, 17 and 18. Suitable libraries can be prepared from human tissue and cell samples. In order to isolate cDNA encoding a polymorphic human AKAP10 libraries prepared from different tissues can be screend since the allele can not be expressed in all tissues or at similar levels in different tissues. The library can be screened with a portion of DNA including substantially the entire human AKAP10 or polymorphic AKAP10 protein-encoding sequence as set forth in SEQ. ID. NOS. 1, 3, 13, 14, 17 and 18, or the library can be screened with a suitable probe.

After screening the library, positive clones are identified by detecting a hybridization signal; the identified clones are characterized by restriction enzyme mapping and/or DNA sequence analysis, and then examined, by comparison with the sequences set forth herein to ascertain whether they include DNA encoding a complete polymorphic human AKAP10 protein (i.e., if they include translation initiation and termination codons). If the selected clones are incomplete, they can be used to rescreen the same or a different library to obtain overlapping clones. If the library is genomic, then the overlapping clones can include exons and introns. If the library is a cDNA library, then the overlapping clones will include an open reading frame. In both instances, clones can be identified by comparison with the DNA and encoded proteins provided herein.

In an alternative method, oligonucleotides based on the human AKAP10 or polymorphic AKAP10 protein-encoding sequence as set forth in SEQ. ID. NOS. 1, 3, 13, 14, 17 and 18, can be used to amplify fragments of the protein coding region of the AKAP10 gene from human cDNA or genomic sequence.

The isolated nucleic acid sequences can be incorporated into vectors for further manipulation. As used herein, vector (or plasmid) refers to discrete elements that are used to introduce heterologous DNA into cells for either expression or replication thereof. Selection and use of such vehicles are well within the skill of the artisan.

K. Transgenic Animals

Methods for making transgenic animals using a variety of transgenes have been described in Wagner et al., Proc. Nat. Acad. Sc. U.S.A., Vol. 78, p. 5016, 1981; Stewart et al., Science, Vol. 217, p. 1046, 1982; Constantini et al, Nature, Vol. 294, p. 92, 1981; Lacy et al., Cell, Vol. 34, p. 343, 1983; McKnight et al., Cell, Vol. 34, p. 335, 1983; Brinstar et al., Nature, Vol. 306, p. 332, 1983; Palmiter et al., Nature, Vol. 300, p. 611, 1982; Palmiter et al., Cell, Vol. 29, p. 701, 1982 and Palmiter et al., Science, Vol. 222, p. 809, 1983. Such methods are described in U.S. Pat. Nos. 6,175,057; 6,180,849 and 6,133,502.

The term “transgene” is used herein to describe genetic material that has been or is about to be artificially inserted into the genome of a mammalian cell, particularly a mammalian cell of a living animal. The transgene is used to transform a cell, meaning that a permanent or transient genetic change, typically a permanent genetic change, is induced in a cell following incorporation of exogenous DNA. A permanent genetic change is generally achieved by introduction of the DNA into the genome of the cell. Vectors for stable integration include, but are not limited to, plasmids, retroviruses and other animal viruses and YACS. Of interest are transgenic mammals, including, but are not limited to, cows, pigs, goats, horses and others, and particularly rodents, including rats and mice. Typically, the transgenic-animals are mice.

Transgenic animals contain an exogenous nucleic acid sequence present as an extrachromosomal element or stably integrated in all or a portion of its cells, especially germ cells. Unless otherwise indicated, it will be assumed that a transgenic animal comprises stable changes to the germline sequence. During the initial construction of the animal, “chimeras” or “chimeric animals” are generated, in which only a subset of cells have the altered genome. Chimeras are primarily used for breeding purposes in order to generate the desired transgenic animal. Animals having a heterozygous alteration are generated by breeding of chimeras. Male and female heterozygotes are typically bred to generate homozygous animals.

The exogenous gene is usually either from a different species than the animal host, or is otherwise altered in its coding or non-coding sequence. The introduced gene can be a wild-type gene, naturally occurring polymorphism or a genetically manipulated sequence, for example having deletions, substitutions or insertions in the coding or non-coding regions. When the introduced gene is a coding sequence, it is usually operably linked to a promoter, which can be constitutive or inducible, and other regulatory sequences required for expression in the host animal.

Transgenic animals can comprise other genetic alterations in addition to the presence of alleles of AKAP genes. For example, the genome can be altered to affect the function of the endogenous genes, contain marker genes, or contain other genetic alterations (e.g., alleles of other genes associated with cardiovascular disease).

A “knock-out” of a gene means an alteration in the sequence of the gene that results in a decrease of function of the target gene, typically such that target gene expression is undetectable or insignificant. A knock-out of an endogenous AKAP gene means that function of the gene has been substantially decreased so that expression is not detectable or only present at insignificant levels. “Knock-out” transgenics can be transgenic animals having a heterozygous knock-out of an AKAP gene or a homozygous knock-out. “Knock-outs” also include conditional knock-outs, where alteration of the target gene can occur upon, for example, exposure of the animal to a substance that promotes target gene alteration, introduction of an enzyme that promotes recombination at the target gene site (e.g., Cre in the Cre-lox system), or other method for directing the target gene alteration postnatally.

A “knock-in” of a target gene means an alteration in a host cell genome that results in altered expression (e.g., increased (including ectopic)) of the target gene, e.g., by introduction of an additional copy of the target gene, or by operatively inserting a regulatory sequence that provides for enhanced expression of an endogenous copy of the target gene. “Knock-in” transgenics of interest can be transgenic animals having a knock-in of an AKAP gene. Such transgenics can be heterozygous or homozygous for the knock-in gene. “Knock-ins” also encompass conditional knock-ins.

A construct is suitable for use in the generation of transgenic animals if it allows the desired level of expression of an AKAP encoding sequence or the encoding sequence of another gene associated with cardiovascular disease. Methods of isolating and cloning a desired sequence, as well as suitable constructs for expression of a selected sequence in a host animal, are well known in the art and are described below.

For the introduction of a gene into the subject animal, it is generally advantageous to use the gene as a gene construct wherein the gene is ligated downstream of a promoter capable of and operably linked to expressing the gene in the subject animal cells. Specifically, a transgenic non-human mammal showing high expression of the desired gene can be created by microinjecting a vector ligated with the gene into a fertilized egg of the subject non-human mammal (e.g., rat fertilized egg) downstream of various promoters capable of expressing the protein and/or the corresponding protein derived from various mammals (rabbits, dogs, cats, guinea pigs, hamsters, rats, mice etc., typicall rats etc.) Useful vectors include Escherichia coli-derived plasmids, Bacillus subtilis-derived plasmids, yeast-derived plasmids, bacteriophages such as lambda, phage, retroviruses such as Moloney leukemia virus, and animal viruses such as vaccinia virus or baculovirus.

Useful promoters for such gene expression regulation include, for example, promoters for genes derived from viruses (cytomegalovirus, Moloney leukemia virus, JC virus, breast cancer virus etc.), and promoters for genes derived from various mammals (humans, rabbits, dogs, cats, guinea pigs, hamsters, rats, mice etc.) and birds (chickens etc.) (e.g., genes for albumin, insulin II, erythropoietin, endothelin, osteocalcin, muscular creatine kinase, platelet-derived growth factor beta, keratins K1, K10 and K14, collagen types I and II, atrial natriuretic factor, dopamine beta-hydroxylase, endothelial receptor tyrosine kinase (generally abbreviated Tie2), sodium-potassium adenosine triphosphorylase (generally abbreviated Na,K-ATPase), neurofilament light chain, metallothioneins I and IIA, metalloproteinase I tissue inhibitor, MHC class I antigen (generally abbreviated H-2L), smooth muscle alpha actin, polypeptide chain elongation factor 1 alpha (EF-1 alpha), beta actin, alpha and beta myosin heavy chains, myosin light chains 1 and 2, myelin base protein, serum amyloid component, myoglobin, renin etc.).

In one embodiment, the above-mentioned vectors have a sequence for terminating the transcription of the desired messenger RNA in the transgenic animal (generally referred to as terminator); for example, gene expression can be manipulated using a sequence with such function contained in various genes derived from viruses, mammals and birds. In one example, the simian virus SV40 terminator etc. are commonly used. Additionally, for the purpose of increasing the expression of the desired gene, the splicing signal and enhancer region of each gene, a portion of the intron of a eukaryotic organism gene can be ligated 5′ upstream of the promoter region, or between the promoter region and the translational region, or 3′ downstream of the translational region as desired.

A translational region for a protein of interest can be obtained using the entire or portion of genomic DNA of blood, kidney or fibroblast origin from various mammals (humans, rabbits, dogs, cats, guinea pigs, hamsters, rats, mice etc.) or of various commercially available genomic DNA libraries, as a starting material, or using complementary DNA prepared by a known method from RNA of blood, kidney or fibroblast origin as a starting material. Also, an exogenous gene can be obtained using complementary DNA prepared by a known method from RNA of human fibroblast origin as a starting material. All these translational regions can be used in transgenic animals.

To obtain the translational region, it is possible to prepare DNA incorporating an exogenous gene encoding the protein of interest in which the gene is ligated downstream of the above-mentioned promoter (typically upstream of the translation termination site) as a gene construct capable of being expressed in the transgenic animal.

DNA constructs for random integration need not include regions of homology to mediate recombination. Where homologous recombination is desired, the DNA constructs will comprise at least a portion of the target gene with the desired genetic modification, and will include regions of homology to the target locus. Conveniently, markers for positive and negative selection are included. Methods for generating cells having targeted gene modifications through homologous recombination are known in the art. For various techniques for transfecting mammalian cells, see Keown et al. (1990) Methods in Enzymology 185:527-537.

The transgenic animal can be created by introducing an AKAP gene construct into, for example, an unfertilized egg, a fertilized egg, a spermatozoon or a germinal cell containing a primordial germinal cell thereof, typically in the embryogenic stage in the-development of a non-human mammal (more typically in the single-cell or fertilized cell stage and generally before the 8-cell phase), by standard means, such as the calcium phosphate method, the electric pulse method, the lipofection method, the agglutination method, the microinjection method, the particle gun method, the DEAE-dextran method and other such method. Also, it is possible to introduce a desired AKAP gene into a somatic cell, a living organ, a tissue cell or other cell, by gene transformation methods, and use it for cell culture, tissue culture and any other method of propagation. Furthermore, these cells can be fused with the above-described germinal cell by a commonly known cell fusion method to create a transgenic animal.

For embryonic stem (ES) cells, an ES cell line can be employed, or embryonic cells can be obtained freshly from a host, e.g. mouse, rat, guinea pig, etc. Such cells are grown on an appropriate fibroblast-feeder layer or grown in the presence of appropriate growth factors, such as leukemia inhibiting factor (LIF). When ES cells have been transformed, they can be used to produce transgenic animals. After transformation, the cells are plated onto a feeder layer in an appropriate medium. Cells containing the construct can be detected by employing a selective medium. After sufficient time for colonies to grow, they are picked and analyzed for the occurrence of homologous recombination or integration of the construct. Those colonies that are positive can then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting litters screened for mutant cells having the construct. By providing for a different phenotype of the blastocyst and the ES cells, chimeric progeny can be readily detected. The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in in vitro culture.

Animals containing more than one transgene, such as allelic variants of AKAP genes and/or other genes associated with morbidity and/or mortality can be made by sequentially introducing individual alleles into an animal in order to produce the desired phenotype (manifestation of morbidity and/or predisposition to early mortality).

L. Screening Assays for Modulators

Modulators of AKAP10 biological activities can be identified by using any of the disclosed methods related to AKAP10 binding to PKA, AKAP10 localization in the mitochondria, binding to other signaling enzymes and phosphorylation by PKA.

In particular, once a variant protein such as AKAP10-5 is contacted with a potential modulating molecule the effect of the molecule on the binding between AKAP protein and PKA can be determined by using the assays disclosed in the section entitled “Effect of Allelic Variants”. For example mitochondria can be isolated from cells exposed to the potential modulating molecule. PKA protein can then be isolated and quantitated or phosphorylation can be determined using the disclosed PKA assay. An increase in the amount of PKA protein in the mitochondria or the quantity of test peptide phosphorylated by mitochondrial isolated PKA would indicate a positive effect of the test molecule. Binding of AKAP10 protein and PKA could be directly assessed using an in vitro binding assay, or other disclosed binding assays, or by immunoassays such as immunoprecipitation.

For allelic variants that do not alter the AKAP10 protein the effect of a potential modulating molecule can be assayed by examining PKA RNA using the various methods disclosed for RNA analysis.

M. Ribozymes

A ribozyme targets the RNA genome and RNA transcripts and copies thereof. Each ribozyme molecule contains a catalytically active segment capable of cleaving the plus or minus strand of RNA, and further comprises flanking sequences having a nucleotide sequence complementary to portions of the target RNA. The flanking sequences serve to anneal the ribozyme to the RNA in a site-specific manner. Absolute complementarity of the flanking sequences to the target sequence is not necessary, however, as only an amount of complementarity sufficient to form a duplex with the target RNA and to allow the catalytically active segment of the ribozyme to cleave at the target sites is necessary. Thus, only sufficient complementarity to permit the ribozyme to be hybridizable with the target RNA is required. In some embodiments of the present invention the enzymatic RNA molecule is formed in a hammerhead motif but the ribozyme can also be formed in the motif of a hairpin, hepatitis delta virus, group I intron or RNAse P RNA (in association with an RNA guide sequence). Examples of hammerhead motifs are described by Rossi et al., AIDS Res. Hum. Retrovir. 8:183 (1992), hairpin motifs are described by Hampel et al., Biochem. 28:4929 (1989) and Hampel et al., Nucl. Acids Res. 18:299 (1990), the hepatitis delta virus motif is exemplified in Perrotta and Been, Biochem. 31:16 (1992), an RNAseP motif is described in Gueerier-Takada et al., Cell 35:849 (1983), and examples of the group I intron motif are described in Cech et al., U.S. Pat. No. 4,987,071, each of the foregoing disclosures being incorporated herein by reference.

Ribozymes can be prepared by chemical synthesis or produced by recombinant vectors according to methods established for the synthesis of RNA molecules. See, e.g., Sambrook et al., Molecular Cloning, A Laboratory Manual, 2d ed., Cold Spring Harbor Laboratory Press, Cold Spring Harbor, N.Y. (1989), incorporated herein by reference. The ribozyme sequence can be synthesized, for example, using RNA polymerases such as T7 or SP6. The ribozymes can be prepared from a corresponding DNA sequence (DNA which on transcription yields a ribozyme) operably linked to an RNA polymerase promoter such as the promoter for T7 RNA polymerase or SP6 RNA polymerase. A DNA sequence corresponding to a ribozyme can be ligated in to a DNA vector, such as a plasmid, bacteriophage or other virus. Where the transfer vector contains an RNA polymerase promoter operably linked to DNA corresponding to a ribozyme, the ribozyme can be conveniently produced upon incubation with an RNA polymerase. Ribozymes can therefore be produced in vitro by incubation of RNA polymerase with an RNA polymerase promoter operably linked to DNA corresponding to a ribozyme, in the presence of ribonucleotides. In vivo, procaryotic or eucaryotic cells (including mammalian cells) can be transfected with an appropriate vector containing genetic material corresponding to a ribozyme, operably linked to an RNA polymerase promoter such that the ribozyme is transcribed in the host cell. Ribozymes can be directly transcribed in vivo from a transfer vector, or alternatively, can be transcribed as part of a larger RNA molecule. For example, DNA corresponding to ribozyme sequence can be ligated into the 3′ end of a carrier gene, for example, after a translation stop signal. Larger RNA molecules can help to stabilize the ribozyme molecules against nuclease digestion within the cells. On translation the carrier gene can give rise to a protein, whose presence can be directly assayed if desired, for example, by enzymatic reaction when the carrier gene encodes an enzyme.

Those of skill in the art based on the above description and the sequences disclosed herein can design ribozymes to target RNA representing the allelic variants of the AKAP10 gene. For example, the sequence of anti-AKAP10-5 hammerhead ribozyme is 5-U G C A C U G A N G A G C C U G G A C G A A A C U-3′ (SEQ ID NO: 25). The sequence UGCA is complementary to target RNA with C hybridizing to the G nucleotide at position 2073 of SEQ ID NO: 3 of the AKAP10-5 allelic variant. The simplest hammerhead ribozyme must have UG at the 5′ end of the substrate binding site.

N. Kits

Kits can be used to indicate whether a subject is at risk of increased susceptibility to morbidity and/or predisposition for premature or increased or early mortality. The kits can also be used to determine if a subject has a genetic predisposition to a disorder related to signal transduction. This information could be used, e.g., to optimize treatment of such individuals as a particular genotype can be associated with drug response.

The kits comprise a probe or primer which is capable of hybridizing adjacent to or at a polymorphic region of AKAP10 and thereby identifying whether the AKAP10 gene contains an allelic variant which is associated with increased susceptibility to morbidity and/or predisposition for premature or increased or early mortality or a genetic predisposition to a disorder related to signal transduction and/or protein phosphorylation. The kits further comprise instructions for use in carrying out assays, interpreting results and diagnosing a subject as having increased susceptibility to morbidity and/or predisposition for premature or increased or early mortality or a genetic predisposition to a disorder related to signal transduction and/or protein phosphorylation.

Kits for amplifying a region of AKAP10 gene or other genes associated with morbidity and/or mortality and/or signal transduction comprise two primers which flank a polymorphic region of the gene of interest. For example primers can comprise the sequences of SEQ ID NOs.:5, 6, 7, 10, 12 and 16. For other assays, primers or probes hybridize to a polymorphic region or 5′ or 3′ to a polymorphic region depending on which strand of the target nucleic acid is used. For example, specific probes and primers comprise sequences designated as SEQ ID NOs: 8, 15, 19 and 20. Those of skill in the art can synthesize primers and probes which hybridize adjacent to or at the polymorphic regions described herein and other SNPs in genes associated with morbidity and/or mortality and/or signal transduction

Yet other kits comprise at least one reagent necessary to perform an assay. For example, the kit can comprise an enzyme, such as a nucleic acid polymerase. Alternatively the kit can comprise a buffer or any other necessary reagent.

Yet other kits comprise microarrays of probes to detect allelic variants of the AKAP10 gene. The kits further comprise instructions for their use and interpreting the results.

The following examples are included for illustrative purposes only and are not intended to limit the scope of the invention. The practice of methods and development of the products provided herein employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridizatiion (B. D. Hames & S. J. Higgins eds. 1984); Transcription and Translation (B. D. Hames & S. J. Higgins eds. 1984); Culture of Animal Cells (R. I. Freshney, Alan R. Liss, Inc., 1987); Immobilized Cells and Enzymes (IRL Press, 1986); B. Perbal, A Practical Guide To Molecular Cloning (1984); the treatise, Methods In Enzymology (Academic Press, Inc., New York); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds., Immunochemical Methods In Cell and Molecular Biology (Mayer and Walker, eds., Academic Press, London, 1987); Handbook of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

The following examples are included for illustrative purposes only and are not intended to limit the scope of the invention.

### EXAMPLES

### Example 1

Isolation of DNA from Blood Samples of a Healthy Donor Population

The results are provided of a screen comparing allele frequencies of 6,500 SNPs located in approximately 5,000 genes between a sample of young and elderly healthy individuals. This resulted in the identification of a gene encoding a functional variant with an impact on morbidity that can be involved in the etiology of cardiac dysfunction.

All subjects involved in the studies signed a written informed consent and the institutional ethics committees of participating institutions approved the experimental protocols. Subjects for the disease susceptibility genome screen were part of a sample that was recruited during a routine blood donation from private blood collection centers in San Bernardino and Rancho Mirage, Calif., USA. The staff of the blood collection agencies invited all healthy blood donors to participate, and helped the subjects fill out a consent form and a simple personal data collection form prior to sample collection. The data collection form included information about age, sex, body size, personal and family disease history, and ethnic background of both parents. Information about the identity of the study participants was not recorded. Subjects would be excluded if they failed to meet the blood donation eligibility guidelines established by the American Red Cross. Ethnicity was defined for each subject if they identified both parents as having the same ethnic/geographic background, otherwise they are indicated as “Other”. For the purpose of identifying disease susceptibility associated SNPs, derived from this collection was a discovery cohort consisting of male and female Caucasian-Americans divided into young (18-39 years) and old (>60 years) groups. These groups and others used in this study are shown in Table 4.

TABLE 4
Table 4. Composition of age-, gender-, and ethnicity-stratified
groups. Abbreviations: CA, Caucasian-American; HI, Hispanic-American;
AF, African-American; AS, Asian-American; YF, Young Female;
YM, Young Male; OF, Old Female; OM, Old Male; S.D., Standard
Deviation.

| Group | Gender | N | Age Range | Mean Age (S.D.) | |
|---|---|---|---|---|---|
| CA-YF | Female | 276 | 18-39 | 27.0 | (6.68) |
| CA-YM | Male | 276 | 18-39 | 27.1 | (6.65) |
| CA-OF | Female | 184 | 60-69 | 64.1 | (2.84) |
| CA-OM | Male | 367 | 60-79 | 66.7 | (4.78) |
| HL-YF | Female | 359 | 18-39 | 29.0 | (6.42) |
| HI-YM | Male | 173 | 18-39 | 28.9 | (6.67) |
| HI-OF | Female | 61 | 50-78 | 55.6 | (5.66) |
| HI-OM | Male | 64 | 50-89 | 57.9 | (7.49) |
| AF | Female/Male | 97/97 | 18-76 | 37.4 | (11.49) |
| AS | Female/Male | 62/64 | 18-65 | 34.4 | (12.18) |

A follow-up study of the SNPs significantly associated with age in the genome scan was carried out in a sample of 417 Caucasian twin pairs from the adult twin registry at St Thomas Hospital, London, United Kingdom. Participants in this collection were enrolled without regard to health status as previously described (Andrew et al., 2001, Twin Res., 4:464-477). For this study, 97 traits were selected to explore possible disease associations. The selected traits have connections to many disease areas, including cardiovascular diseases, diabetes, hypertension, obesity, and osteoporosis.

Practically a healthy subject, when human, is defined as human donor who passes blood bank criteria to donate blood for eventual use in the general population. These criteria are as follows: free of detectable viral, bacterial, mycoplasma, and parasitic infections; not anemic; and then further selected based upon a questionnaire regarding history. Thus, a healthy population represents an unbiased population of sufficient health to donate blood according to blood bank criteria, and not further selected for any disease state. Typically such individuals are not taking any medications.

Blood was obtained from a donor by venous puncture and preserved with 1 mM EDTA pH 8.0. Ten milliliters of whole blood from each donor was centrifuged at 2000× g. One milliliter of the buffy coat was added to 9 milliliters of 155 mM NH₄Cl, 10 mM KHCO₃, and 0.1 mM Na₂EDTA, incubated 10 minutes at room temperature and centrifuged for 10 minutes at 2000× g. The supernatant was removed and the white cell pellet was washed in 155 mM NH₄Cl, 10 mM KHCO₃, and 0.1 mM Na₂EDTA and resuspended in 4.5 milliliters of 50 mM Tris, 5 mM EDTA, and 1% SDS. Proteins were precipitated from the cell lysate by 6M ammonium acetate pH 7.3 and separated from the nucleic acid by centrifugation at 3000× g. The nucleic acid was recovered from the supernatant by the addition of an equal volume of 100% isopropanol and centrifugation at 2000× g. The dried nucleic acid pellet was hydrated in 10 mM Tris pH 7.6 and 1 mM Na₂EDTA and stored at 4° C.

Statistical Analysis

Estimates of allele frequencies derived from pooled DNA were based on independent mass spectrometry measurements of four analyte aliquots derived from a single PCR reaction. The median standard deviation for these values was approximately 0.01. For comparing allele frequencies between the young and old pools, females and males were analyzed separately. The statistic used to test the difference in allele frequencies between pools was of the form:

$$Z = \frac{{\hat{p}}_{y} - {\hat{p}}_{o}}{{( {\hat{p}}_{y} ( 1 - {\hat{p}}_{y} ) / 2 {n}_{y} + ( {\hat{p}}_{o} ) / {2}_{o}^{n} + {ε}_{y}^{2} + {ε}_{o}^{2} )}^{1 / 2}} ,$$

which follows a standard normal distribution. Here \hat{p}_{y} and \hat{p}ₒ are the allele frequency estimates and ε_{y}² and εₒ² are estimates of measurement variability calculated from measurement replicates in the young and old pools, respectively. In this study, no correction was made for additional sources of variation or for multiple testing. Rather, SNPs were identified that had p-values less than 0.05 among all measured SNPs, followed by a second, independent measurement of all significant SNPs based on three separate PCRs of each DNA pool. The results of the second round of measurement were analyzed in a manner similar to the first round, and were compared for consistency. SNPs that showed statistically significant differences between young and old groups from pooled DNA analyses were individually genotyped for final validation.

Estimates of allele frequencies using individual genotype data were found using the gene counting method. Comparisons of allele frequencies as well as genotype frequencies between groups were carried out using a chi-squared test of independence.

The SNPs found to be associated with age were further analyzed for association with disease-related quantitative traits in the twin collection. The analysis was conducted using a quantitative transmission-disequilibrium test (QTDT) as described by Abecasis et al., 2000, Am J Hum Genet., 66:279-292 to take advantage of the twin-based sample and to control for admixture and other non-genetic sources of variation. The form of the test was implemented that does not require the estimation of variance components. Formal statistical procedures to account for multiple testing were not use, but the distribution of the resulting p-values is reported.

Results of Genome-Wide SNP Analysis for Morbidity Gene Discovery

A strategy was pursued that utilizes estimates of allele frequencies in DNA pools to screen large numbers of SNPs. To apply this to disease susceptibility gene discovery, the Caucasian-American individuals were divided by age (under 40 and over 60 years) and by sex (Table 4). The fraction of included subjects reporting any health problem was only 1.8% and 2.9% in young females and males, and 3.8% and 3.5% in old females and males, respectively. In this study, a collection was used of 6,500 exonic SNPs located in approximately 5,000 genes or expressed sequence tag (EST) clusters. The majority of assays for these SNPs, originally identified in an in silico discovery project, were developed in collaboration with the National Cancer Institute (NCI).

Identification of D-AKAP2 as a Candidate Gene

More than 50 markers were identified out of the 6,500 tested markers that show a reproducibly significant allele frequency change between the two age groups in at least one gender (P<0.05). The SNP that demonstrated the strongest association with age in both genders is located within the D-AKAP2 gene.

D-AKAP2 codes for dual-specific A-kinase anchor protein 2, which is part of a family of scaffold proteins known as A-kinase anchoring proteins (AKAPs). AKAPs bind the regulatory subunit of cAMP-dependent Protein Kinase (PKA), and target the kinase to various intracellular locations, localizing cAMP-mediated activation of the kinase. PKA is a broad specificity kinase and phosphorylates numerous proteins that function in many essential cellular processes such as metabolism, gene transcription, cell division, and neuronal transmission. In the inactive state, PKA is a tetramer consisting of two catalytic (C) and two regulatory (R) subunits. The dual specificity of D-AKAP2 is defined by its ability to bind both the RI and RII isoforms of PKA.

An A>G polymorphism in the 3′ untranslated region (3′UTR) of D-AKAP2 showed a significant decrease of about 8% (P<0.01) of the G allele in the older sample of both genders (Table 5). The marker was individually genotyped and the frequency differences between young and old individuals calculated from the genotypes were very similar to the pool results. There was a slight skewing of frequencies in the pools likely resulting from uneven PCR amplification of the two alleles. This led to an underestimation of the G allele frequency in all pools but did not impact the significance of the differences between young and old (Table 5).

TABLE 5
Table 5. Comparison of allele frequencies between young
and old groups. Sample sizes are the same as those of
the corresponding groups in Table 2. Allele frequencies
for each SNP are given for the G allele.

Allelle Frequency

| | | | |
|---|---|---|---|
| Young | Old | Difference | P-Value |

Pool Data
3′ UTR

| | | | | |
|---|---|---|---|---|
| Caucasian Female | 0.322 | 0.240 | 0.082 | 0.007 |
| Caucasian Male | 0.362 | 0.232 | 0.076 | 0.002 |

Genotype Data
3′ UTR

| | | | | |
|---|---|---|---|---|
| Caucasian Female | 0.274 | 0.212 | 0.062 | 0.034 |
| Caucasian Male | 0.304 | 0.232 | 0.072 | 0.004 |

1646V

| | | | | |
|---|---|---|---|---|
| Caucasian Female | 0.402 | 0.318 | 0.084 | 0.009 |
| Causian Male | 0.429 | 0.369 | 0.060 | 0.030 |
| Hispanic Female | 0.445 | 0.316 | 0.129 | 0.008 |
| Hispanic Male | 0.436 | 0.375 | 0.061 | 0.229 |

In10

| | | | | |
|---|---|---|---|---|
| Caucasian Female | 0.877 | 0.871 | 0.006 | 0.147 |
| Caucasian Male | 0.880 | 0.865 | 0.015 | 0.360 |

To identify common polymorphisms in the D-AKAP2 gene, the 15 exons and 100-200 bp of the flanking sequence were sequences. The analysis of 36 chromosomes revealed only two additional polymorphisms: An A>G SNP in intron 10, six nucleotides downstream from exon 10 (In10), and an A>G SNP in exon 14 (corresponding to AKAP10-5 at nucleotide 2073 of SEQ ID NO:1), leading to an amino acid substitution Ile to Val at position 646 (I646V). Individual genotyping of the Caucasian-American samples showed that the intron 10 SNP exhibits no morbidity-association. The I646V polymorphism, however, was found to be significantly different between young and old in both males (P=0.03) and females (P=0.009) (Table 5). There was no significant difference between young males and females and between old males and females. The Bayesian, coalescent theory-based method (Stephens et al., 2001, Am J Hum Genet. 68:978-989) was applied to construct haplotypes at these three tightly linked sites for each subject. The estimates of the disequilibrium (D′) between the 3′UTR and the I646V and In10 SNPs were 0.991 and 0.255 (r2=0.55 and 0.03), respectively. The distance between the markers in strong disequilibrium, 3′UTR and I646V which both showed association, is about 4 kb, while the intron 10 SNP is located approximately 23 kb upstream of I646V.

As expected, the changes in genotype frequencies between age groups for the three sites showed a similar level of statistical significance as the changes in allele frequencies. At the 3′UTR and the I646V variable sites, GG homozygotes were reduced and AA homozygotes increased in the older sample population of both genders. This further supports the hypothesis that the G allele, which determines the Val allele at I646V, is associated with a negative health impact.

Hispanic-American samples were also genotyped for the I646V variation. Since there were only a small number of Hispanic-American individuals over 60 years, the older sample population was extended to all individuals older than 50 years of age (Table 4). While females showed a statistically significant allele frequency difference between old and young (P=0.008), the males did not, which can be due to the relatively small number of male individuals (Table 5). The allele frequency differences in both genders were comparable to those observed in Caucasian-Americans. The frequency of the G allele decreased by 0.129 in females and 0.061 in males, the GG homozygote by 0.087 in females and 0.072 in males. These results further support the association of the Val allele with morbidity and/or mortality, and therefore the involvement of this gene in one or several disease processes. Another non-synonymous D-AKAP2 variation retrieved from dbSNP has been verified. The G-A transversion in exon 4 results in an Arg to His substitution at position 249 (R249H; corresponding to a G to A transversion at nucleotide 883 of SEQ ID NO:1 encoding human D-AKAP2). The Arg was found to be in complete linkage disequilibrium with the Ile at position 646, occurring together in every case, and therefore shows the same age effect.

Association of D-AKAP2 Genotypes with a Cardiac Trait

In an effort to identify traits correlated with the observed age association of the I646V SNP, we utilized a cohort of 417 fasting Caucasian twin pairs with extensive coverage for a variety of disease-related traits. Of the 97 traits analyzed, only the PR interval was statistically significant at a nominal level of 0.05. The estimate from the QTDT model of the average effect of the G allele (Val) was to decrease the PR interval 6.3 units (P=0.007). The genotype means in the subset of 207 informative twin pairs were 157±23.4, 152 ±26.9, and 146±25.4 (mean ± standard deviation) for genotypes AA, GA, and GG, respectively at a position corresponding to nucleotide 2073 of SEQ ID NO:1.

### Example 2

Detection of AKAP10-1 by MassEXTEND™ Assay Detection Methods

AKAP10-1 is an allele of the AKAP10 gene with a single nucleotide polymorphism at nucleotide number 156277 (based on the sequence of a genomic clone of the AKAP10 gene, GenBank Accession No. AC005730). The single nucleotide polymorphism is a T to C transversion located in the 3′non-translated region of the gene encoding AKAP10. PCR primers were synthesized by OPERON (Alameda, Calif.) using phosphoramidite chemistry. Amplification of the AKAP10 target sequence was carried out in single 50 μl PCR reaction with 25 ng of human genomic DNA obtained from samples as described in Example 1. Each reaction containing IX PCR buffer (Qiagen, Valencia, Calif.), 200 μM dNTPs, 1 U Hotstar Taq polymerase (Qiagen, Valencia, Calif.), 4 mM MgCl₂, and 25 pmols of the forward primer containing the universal primer sequence and the target specific sequence 5′-TCTCAATCATGTGCATTGAGG-3′ (SEQ ID NO: 5) 2 pmoles of the reverse primer 5′-AGCGGATAACAATTTCACACAGGGATCACACAGCCATCAGCAG-3′ (SEQ ID NO: 6) and 10 pmoles of a biotinylated universal primer complementary to the 5′ end of the PCR amplicon 5′-AGCGGATAACAATTTCACACAGG-3′ (SEQ ID NO: 7). Alternatively, the biotinylated universal primer could be 5′-GGCGCACGCCTCCACG-3′ (SEQ ID NO: 16). After an initial round of amplification of the target with the specific forward and reverse primer, the 5′ biotinylated universal primer was hybridized and acted as a reverse primer thereby introducing a 3′ biotin capture moiety into the molecule. The amplification protocol resulted in a 5′-biotinylated double stranded DNA amplicon, which dramatically reduces the cost of high throughput genotyping by eliminating the need to 5′ biotin label each forward primer used in a genotyping. Thermal cycling was performed in 0.2 mL tubes or 96 well plate using an MJ Research Thermal Cycler (Waltham, Mass.) (calculated temperature) with the following cycling parameters: 94° C. for 5 min; 45 cycles: 94° C. for 20 sec, 56° C. for 30 sec, 72° C. for 60 sec; 72° C. 3 min.

Immobilization of DNA

The 50 μl PCR reaction was added to 25 μl of streptavidin coated magnetic bead (Dynal) prewashed three times and resuspended in 1M NH₄Cl, 0.06M NH₄OH. The PCR amplicons were allowed to bind to the beads for 15 minutes at room temperature. The beads were then collected with a magnet and the supernatant containing unbound DNA was removed. The unbound strand was release from the double stranded amplicons by incubation in 100 mM NaOH and washing of the beads three times with 10 mM Tris pH 8.0.

Genotyping

Genotyping was carried out using the MassEXTEND™ assay and MALDI-TOF. The SNP identified at position 156277 of AKAP10 in the GenBank sequence is represented as a T to C transversion. The MassEXTEND™ assay detected the sequence of the complementary strand at the polymorphic position, thus the primer extension product incorporated either a T or a C. The DNA coated magnetic beads were resuspended in 26 mM Tris-HCL pH 9.5, 6.5 mM MgCl₂ and 50 mM each of dTTPs and 50 mM each of ddCTP, ddATP, ddGTP, 2.5 U of a thermostable DNA polymerase (Amersham Pharmacia Biotech, Piscataway, N.J.) and 20 pmoles of a template specific oligonucleotide primer 5′-CTGGCGCCCACGTGGTCAA-3′ (SEQ ID NO: 8) (Operon, Alameda, Calif.). Primer extension occurs with three cycles of oligonucleotide primer was hybridization and extension. The extension products were analyzed after denaturation from the template with 50 mM NH₄Cl and transfer of 150 nl each sample to a silicon chip preloaded with 150 nl of H3PA (3-hydroxy picolinic acid) (Sigma Aldrich, St. Louis, Mo.) matrix material. The sample material was allowed to crystallize and analyzed by MALDI-TOF (Bruker Daltonics, Billerica, Mass., PerSeptive, Foster City, Calif.). The mass of the primer used in the MassEXTEND™ reaction was 5500.6 daltons. The allelic variant results in the addition of ddC to the primer to produce an extension product having a mass of 5773.8 daltons. The predominant allele is extended by the addition of dT and ddG to the primer to produce an extension product having a mass of 6101 daltons.

The SNP that is present in AKAP10-1 is a T to C transversion at nucleotide number 156277 of the sequence of a genomic clone of the AKAP10 gene (GenBank Accession No. AC005730) (SEQ ID NO: 17). SEQ ID NO:17 represents the nucleotide sequence of human chromosome 17, which contains the genomic nucleotide sequence of the human AKAP10 gene at approximately nucleotide 83,580 to nucleotide 156,577. SEQ ID NO: 18 represents the nucleotide sequence of human chromosome 17, which contains the genomic nucleotide sequence of the human AKAP10-1 allele.

The frequency of the AKAP10-1 allelic variant was measured in a population of age selected healthy individuals. Five hundred fifty-two (552) individuals between the ages of 18-39 years (276 females, 276 males) and 552 individuals between the ages of 60-79 (184 females between the ages of 60-69, 368 males between the age of 60-79) were tested for the presence of the allelic variant localized in the non-translated 3′ region of AKAP 10. Differences in the frequency of this variant with increasing age groups were observed among healthy individuals. Statistical analysis showed that the significance level for differences in the allelic frequency for alleles between the “younger” and the “older” populations was p=0.0009 and for genotypes was p=0.003. Differences between age groups are significant. For the total population allele significance is p=0.0009, and genotype significance is p=0.003.

The young and old populations were in Hardy-Weinberg equilibrium. A preferential change of one particular genotype was not seen.

The polymorphism is localized in the non-translated 3′-region of the gene encoding the human protein kinase A anchoring protein (AKAP10). The gene is located on chromosome 17. Its structure includes 15 exons and 14 intervening sequences (introns). The encoded protein is responsible for the sub-cellular localization of the cAMP-dependent protein kinase and, therefore, plays a key role in the G-protein mediated receptor-signaling pathway (Huang et al. PNAS (1007) 94:11184-11189). Since its localization is outside the coding region, this polymorphism is most likely in linkage disequilibrium (LD) with other non-synonymous polymorphisms that could cause amino acid substitutions and subsequently alter the function of the protein.

### Example 3

Discovery of AKAP10-5 Allele

Genomic DNA was isolated from blood (see Example 1) of seventeen (17) individuals with a genotype CC at the AKAP10-1 gene locus and a single heterozygous individual (CT) (as described in Example 2). A target sequence in the AKAP10-1 gene which encodes the C-terminal PKA binding domain was amplified using the polymerase chain reaction. PCR primers were synthesized by OPERON (Alameda, Calif.) using phosphoramidite chemistry. Amplification of the AKAP10-1 target sequence was carried out in individual 50 μl PCR reaction with 25 ng of human genomic DNA templates. Each reaction containing I X PCR buffer (Qiagen, Valencia, Calif.), 200 μM dNTPs, IU Hotstar Taq polymerase (Qiagen, Valencia, Calif.), 4 mM MgCl₂, and 25 pmols of the forward primer containing the universal primer sequence and the target specific sequence 5′-TCC CAA AGT GCT GGA ATT AC-3′ (SEQ ID NO: 9), 2 pmoles of the reverse primer 5′-GTC CAA TAT ATG CAA ACA GTT G-3′(SEQ ID NO:10). Thermal cycling was performed in 0.2 mL tubes or 96 well plate using an MJ Research Thermal Cycler (MJ Research, Waltham, Mass.) (calculated temperature) with the following cycling parameters: 94° C. for 5 min; 45 cycles; 94° C. for 20 sec, 56° C. for 30 sec, 72° C. for 60 sec; 72° C. 3 min. After amplification the amplicons were purified by chromatography (Mo Bio Laboratories (Solana Beach, Calif.).

The sequence of the 18 amplicons, representing the target region, was determined using a standard Sanger cycle sequencing method with 25 nmoles of the PCR amplicon, 3.2 μM DNA sequencing primer 5′-CCC ACA GCA GTT AAT CCT TC-3′ (SEQ ID NO:11) and chain terminating dRhodamine labeled 2′, 3′ dideoxynucleotides (PE Biosystems, Foster City, Calif.) using the following cycling parameters: 96° C. for 15 sec, 25 cycles: 55° C. for 15 sec, 60° C. for 4 min. The sequencing products were precipitated by 0.3M NaOAc and ethanol, the precipitate was centrifuged and dried. The pellets were resuspended in deionized formamide and separated on a on a 5% polyacrylamide gel. The sequence was determined using the “Sequencher” software (Gene Codes, Ann Arbor, Mich.).

The sequence of all 17 of the amplicons which are homozygous for the AKAP10-1 SNP revealed a polymorphism at nucleotide position 152171 (numbering for GenBank Accession No. AC005730 for AKAP10 genomic clone) with A replaced by G. This SNP can also be designated as located at nucleotide 2073 of a cDNA clone of the wildtype AKAP10 (SEQ ID NO:1) (GenBank Accession No. AF037439). This single nucleotide polymorphism was designated as AKAP10-5 (SEQ ID NO:3) and results in a substitution of a valine for an isoleucine residue at amino acid position 646 (SEQ ID NO:4).

### Example 4

PCR Amplification and MassEXTEND™ Assay Detection of AKAP10-5 in a Healthy Donor Population

A healthy population stratified by age is a very efficient and a universal screening tool for morbidity associated genes by allowing for the detection of changes of allelic frequencies in the young compared to the old population. Individual samples of this healthy population base can be pooled to further increase the throughput.

Healthy samples were obtained through the blood bank of San Bernardino, Calif. Both parents of the blood donors were of Caucasian origin. Practically a healthy subject, when human, is defined as human donor who passes blood bank criteria to donate blood for eventual use in the general population. These criteria are as follows: free of detectable viral, bacterial, mycoplasma, and parasitic infections; not anemic; and then further selected based upon a questionnaire regarding history. Thus, a healthy population represents an unbiased population of sufficient health to donate blood according to blood bank criteria, and not further selected for any disease state. Typically such individuals are not taking any medications.

PCR primers were synthesized by OPERON (Alameda, Calif.) using phosphoramidite chemistry. Amplification of the AKAP10 target sequence was carried out in single 50 μl PCR reaction with 100 ng-1 ug of pooled human genomic DNAs in a 50 μl PCR reaction. Individual DNA concentrations within the pooled samples were present in equal concentration with the final concentration ranging from 1-25ng. Each reaction contained 1× PCR buffer (Qiagen, Valencia, Calif.), 200 μM dNTPs, 1 U Hotstar Taq polymerase (Qiagen, Valencia, Calif.), 4 mM MgCl₂, and 25 pmols of the forward primer containing the universal primer sequence and the target specific sequence 5′-AGCGGATAACAATTTCACACAGGGAGCTAGCTTGGAAGATTGC-3′ (SEQ ID NO:12), 2 pmoles of the reverse primer 5′-GTCCAATATATGCAAACAGTTG-3′ (SEQ ID NO: 10) and 10 pmoles of a biotinylated universal primer complementary to the 5′ end of the PCR amplicon BIO:5′-AGCGGATAACAATTTCACACAGG-3′ (SEQ ID NO: 7). After an initial round of amplification with the target with the specific forward and reverse primer, the 5′ biotinylated universal primer can then hybridized and acted as a forward primer thereby introducing a 5′ biotin capture moiety into the molecule. The amplification protocol resulted in a 5′-biotinylated double stranded DNA amplicon and dramatically reduces the cost of high throughput genotyping by eliminating the need to 5′ biotin label every forward primer used in a genotyping.

Thermal cycling was performed in 0.2 mL tubes or 96 well plate using an MJ Research Thermal Cycler (Waltham, Mass.) (calculated temperature) with the following cycling parameters: 94° C. for 5 min; 45 cycles: 94° C. for 20 sec, 56° C. for 30 sec; 72° C. for 60 sec; 72° C. 3 min.

Immobilization of DNA

The 50 μl PCR reaction was added to 25 μl of streptavidin coated magnetic beads (Dynal, Oslo, Norway) (Lake Success, N.Y.), which were prewashed three times and resuspended in 1M NH₄Cl, 0.06M NH₄OH. The 5′ end of one strand of the double stranded PCR amplicons were allowed to bind to the beads for 15 minutes at room temperature. The beads were then collected with a magnet and the supernatant containing unbound DNA was removed. The hybridized but unbound strand was released from the double stranded amplicons by incubation in 100 mM NaOH and washing of the beads three times with 10 mM Tris pH 8.0.

Genotyping

The identity of the nucleotide present at the polymorphic site of AKAP 10-5 was determined by using the MassEXTEND™ assay and MALDI-TOF (see, U.S. Pat. No. 6,043,031). The MassEXTEND™ assay is a primer extension assay that utilizes a primer that hybridizes adjacent to the polymorphic region and which is extended in the presence of one or more ddNTPs. Extension is stopped by the incorporation of a dideoxy nucleotide. At a polymorphic site the different alleles produce different length extension products, which are distinguishable by mass spectrometry.

The MassEXTEND™ assay detected the sequence of the sense strand and resulted in the incorporation of either T or C into the extension product. The DNA coated magnetic beads were suspended in 26 mM Tris-HCL pH 9.5; 6.5 mM, MgCl₂ and 50 mM each of dTTPs and 50 mM each of ddCTP, ddATP, ddGTP, 2.5 U of a thermostable DNA polymerase (Amersham Pharmacia Biotech, Piscataway N.J.) and 20 pmoles of a template specific oligonucleotide primer 5′-ACTGAGCCTGCTGCATAA-3′ (SEQ ID NO:15) (Operon) (Alameda, Calif.). Primer extension occurs with three cycles of oligonucleotide primer hybridization and extension. The extension products were analyzed after denaturation from the template with 50 mM NH₄Cl and transfer of 150 nl each sample to a silicon chip preloaded with 150 nl of H3PA (3-hydroxy picolinic acid) (Sigma Aldrich, St. Louis, Mo.) matrix material. The sample material was allowed to crystallize and analyzed by MALDI-TOF (Bruker Daltonics, Billerica, Mass., PerSeptive, Foster City, Calif.). The primer had a mass of 5483.6 daltons. The allelic variant resulted in the addition of a ddC to the primer to produce an extension product having a mass of 5756.8 daltons. The predominant allele resulted in the addition a T and ddG to the primer giving an extension product with a mass of 6101 daltons.

### Example 5

Discovery of AKAP10-7

Genomic DNA isolation, amplification of the target regions and sequencing of amplicons was carried out as in Example 3. Using the sequence of the cDNA for AKAP10, chromosome 17 was BLAST searched to identify the number of exons. Sanger sequencing of the regions around and containing the exons was performed and resulted in the discovery of AKAP10-7 polymorphic region. For AKAP10-7 the forward sequencing primer was CACTGCACCCAGCCTTATG (SEQ ID NO: 23) and the reverse sequencing primer was CTGGGATGTGAAGGAAAGGA (SEQ ID NO: 24).

### Example 6

MassEXTEND™ Assay Detection of AKAP10-7

Samples are obtained and amplified as in Example 4.

The identity of the nucleotide present at the polymorphic site of AKAP 10-7 is determined by using the MassEXTEND™ assay and MALDI-TOF (see, U.S. Pat. No. 6,043,031). The MassEXTEND™ assay detects the sequence of the complementary strand and resulted in the incorporation of either G or A into the extension product. Reactions are carried out as in Example 4. The template specific oligonucleotide primer 5′-CTCTGCGTCTCAGGTATT-3′ (SEQ ID NO: 20). (Operon, Alameda, Calif.). The primer has a mass of 5456.6 daltons. The allelic variant results in the addition of a ddA to the primer to produce an extension product having a mass of 5753.6 daltons. The predominant allele results in the addition a G and ddA to the primer giving an extension product with a mass of 6083.0 daltons.

Since modifications will be apparent to those of skill in this art, it is intended that this invention be limited only by the scope of the appended claims.

1. A method for indicating increased susceptibility of a subject to a disease or disorder, comprising:
conducting an EKG examination;
determining the EKG-PR-interval in the subject, wherein, if the EKG-PR-interval is decreased, then
determining the amino acid present in the subject at position 646 of AKAP10/D-AKAP2 (SEQ ID NO:2) or the nucleotide present at position corresponding to nucleotide 2073 of SEQ ID NO: 1, wherein the presence of Val at position 646 of SEQ ID NO:2 or the presence of a -G- at nucleotide position 2073 of SEQ ID NO: 1, indicates increased susceptibility to a disease or disorder.

2. The method of claim 1, wherein the disease or disorder is selected from the group consisting of cardiovascular disorders, cardiac disease, proliferative disorders, neurological disorders, neurodegenerative disorders, obesity, diabetes and peripheral retinopathies.

3. The method of claim 1, wherein the EKG-PR-interval in the subject is compared to a predetermined age-matched standard EKG-PR-interval.

4. The method of claim 3, wherein the predetermined standard EKG-PR-interval is obtained from a known age-matched control group that is homozygous -AA- at a position corresponding to nucleotide 2073 of SEQ ID NO: 1 or homozygous Ile/Ile at a position corresponding to position 646 of SEQ ID NO:2.

5. The method of claim 3, wherein the predetermined standard EKG-PR-interval is obtained from a known age-matched control group that is heterozygous -GA- at a position corresponding to nucleotide 2073 of SEQ ID NO:1 or heterozygous Val/Ile at a position corresponding to position 646 of SEQ ID NO:2.

6. The method of claim 3, wherein the predetermined standard EKG-PR-interval is obtained from a known age-matched control group that is selected from either homozygous -AA- at a position corresponding to nucleotide 2073 of SEQ ID NO:1 or homozygous Ile/Ile at a position corresponding to position 646 of SEQ ID NO:2; or heterozygous -GA- at a position corresponding to nucleotide 2073 of SEQ ID NO:1 or heterozygous Val/Ile at a position corresponding to position 646 of SEQ ID NO:2.

7. The method of claim 3, wherein the predetermined standard EKG-PR-interval is obtained from a control age-matched subject without heart disease.

8. A method for indicating increased susceptibility of a subject to a disease or disorder associated with the cardiovascular system, comprising:
conducting an EKG exam;
determining the EKG-PR-interval in the subject, wherein, if the EKG-PR-interval is decreased, then
determining the amino acid present at position 646 of AKAP10/D-AKAP2 (SEQ ID NO:2) or the nucleotide present at position corresponding to nucleotide 2073 of SEQ ID NO:1, wherein the presence of Val at position 646 of SEQ ID NO:2 or the presence of a -G- at nucleotide position 2073 of SEQ ID NO:1, indicates increased susceptibility to a disease or disorder associated with the cardiovascular system.

9. The method of claim 8, wherein the EKG-PR-interval in the subject is compared to a predetermined age-matched standard EKG-PR-interval.

10. The method of claim 9, wherein the predetermined standard EKG-PR-interval is obtained from a known age-matched control that is homozygous -AA- at a position corresponding to nucleotide 2073 of SEQ ID NO:1 or homozygous Ile/Ile at a position corresponding to position 646 of SEQ ID NO:2.

11. The method of claim 9, wherein the predetermined standard EKG-PR-interval is obtained from a known age-matched control group that is heterozygous -GA- at a position corresponding to nucleotide 2073 of SEQ ID NO: 1 or heterozygous Val/Ile at a position corresponding to position 646 of SEQ ID NO:2.

12. The method of claim 9, wherein the predetermined standard EKG-PR-interval is obtained from a known age-matched control group that is selected from either homozygous -AA- at a position corresponding to nucleotide 2073 of SEQ ID NO: 1 or homozygous Ile/Ile at a position corresponding to position 646 of SEQ ID NO:2; or heterozygous -GA- at a position corresponding to nucleotide 2073 of SEQ ID NO: 1 or heterozygous Val/Ile at a position corresponding to position 646 of SEQ ID NO:2.

13. The method of claim 9, wherein the predetermined standard EKG-PR-interval is obtained from a control age-matched subject without heart disease.

14-23. (canceled)

24. The method of claim 1, wherein the disease or disorder is selected from one or more of the group consisting of: cardiac arrhythmia, brachycardia, atrial fibrillation, sick sinus syndrome, sudden cardiac arrest, ventricular arrhythmia, ventricular fibrillation, ventricular tachycardia, Wolf-Parkinson-White (WPW) Syndrome, Lown-Ganong-Levin (LGL) Syndrome, hypertension.

25. The method of claim 1, further comprising monitoring the subject for cardiovascular disease.

26. The method of claim 1, further comprising administering to the subject prophylactic steps.

27-29. (canceled)

30. A method for determining responsiveness of a subject to one or more β-blocking agents, comprising:
detecting for the subject the presence or absence of Val at position 646 of SEQ ID NO:2 or a -G- nucleotide at a position corresponding to position 2073 of SEQ ID NO: 1, wherein the presence of a Val at position 646 of SEQ ID NO:2 or a -G- at nucleotide 2073 of SEQ ID NO: 1, is indicative of an increased likelihood that a subject has a modulated response to one or more β-blocking agents compared to a subject who does not have the allelic variant.

31. The method of claim 30, wherein the modulated response is a decreased response to one or more β-blocking agents compared to a subject who does not have the allelic variant.

32. The method of claim 31, wherein the decreased response is a non-response to one or more β-blocking agents compared to a subject who does not have the allelic variant.

33. The method of claim 30, wherein the modulated response is an increased response to one or more β-blocking agents compared to a subject who does not have the allelic variant.

34. The method of claim 30, wherein at least one β-blocking agent is an antagonist of a β-adrenergic receptor.

35. The method of claim 30, wherein at least one β-blocking agent is an agonist of a β-adrenergic receptor.

36. A method for determining responsiveness of a subject to one or more β-blocking agents, comprising:
detecting the presence or absence of Val at position 646 of SEQ ID NO:2 or a -G- nucleotide at a position corresponding to position 2073 of SEQ ID NO: 1, wherein the presence of a Val at position 646 of SEQ ID NO:2 or a -G- at nucleotide 2073 of SEQ ID NO:1, is indicative of an increased likelihood that a subject has an increased response to one or more β-blocking agents compared to a subject who does not have the allelic variant.

37. The method of claim 36, wherein at least one β-blocking agent is an antagonist of a β-adrenergic receptor.

38. The method of claim 36, wherein at least one β-blocking agent is an agonist of a β-adrenergic receptor.

39. A method for determining responsiveness of a subject to one or more β-blocking agents, comprising:
detecting for the subject the presence or absence of Val at position 646 of SEQ ID NO:2 or a -G- nucleotide at a position corresponding to position 2073 of SEQ ID NO: 1, wherein the presence of a Val at position 646 of SEQ ID NO:2 or a -G- at nucleotide 2073 of SEQ ID NO:1, is indicative of an increased likelihood that a subject is non-responsive to one or more β-blocking agents compared to a subject who does not have the allelic variant.

40. The method of claim 39, wherein at least one β-blocking agent is an antagonist of a β-adrenergic receptor.

41. The method of claim 39, wherein at least one β-blocking agent is an agonist of a β-adrenergic receptor.

42. A method for determining responsiveness of a subject to one or more β-blocking agents, comprising:
detecting the presence or absence of Val at position 646 of SEQ ID NO:2 or a -G- nucleotide at a position corresponding to position 2073 of SEQ ID NO: 1, wherein the presence of a Val at position 646 of SEQ ID NO:2 or a -G- at nucleotide 2073 of SEQ ID NO:1, is indicative of an increased likelihood that a subject is hyper-responsive to one or more β-blocking agents compared to a subject who does not have the allelic variant.

43. The method of claim 42, wherein the β-blockers is an antagonist of a β-adrenergic receptor.

44. The method of claim 42, wherein the β-blockers is an agonist of a β-adrenergic receptor.

45. A method for indicating susceptibility to morbidity, increased or early mortality, or morbidity and increased or early mortality of a subject; comprising:
conducting an EKG exam;
determining the EKG-PR-interval in the subject, wherein if the EKG-PR-interval is decreased; then
determining the amino acid at position 646 of AKAP10/D-AKAP2 (SEQ ID NO:2) or the nucleotide present at position corresponding to nucleotide 2073 of SEQ ID NO:1, wherein the presence of Val at position 646 of SEQ ID NO:2 or the presence of a -G- at nucleotide position 2073 of SEQ ID NO:1, indicates increased susceptibility to morbidity, increased or early mortality, or morbidity and increased or early mortality of a subject.

46. The method of claim 45, wherein the EKG-PR-interval in the subject is compared to a predetermined standard EKG-PR-interval.

47. The method of claim 46, wherein the predetermined standard EKG-PR-interval is obtained from a known age-matched control that is homozygous -AA- at a position corresponding to nucleotide 2073 of SEQ ID NO: 1 or homozygous Ile/Ile at a position corresponding to position 646 of SEQ ID NO:2.

48. (canceled)

49. The method of claim 1, wherein the amino acid or nucleotide determining step comprises mass spectrometry.

50. The method of claim 1, wherein the amino acid or nucleotide determining step is effected by detecting a signal moiety selected from the group consisting of radioisotopes, enzymes, antigens, antibodies, spectrophotometric reagents, chemiluminescent reagents, fluorescent reagents and other light producing reagents.

51. The method of claim 1, wherein the subject is heterozygous -GA- at a position corresponding to nucleotide 2073 of SEQ ID NO:1 or heterozygous Val/Ile at a position corresponding to position 646 of SEQ ID NO:2.

52. The method of claim 1, wherein the subject is homozygous -GG- at a position corresponding to nucleotide 2073 of SEQ ID NO:1 or homozygous Val/Val at a position corresponding to position 646 of SEQ ID NO:2.

53. A method for indicating increased susceptibility of a subject to a disease or disorder, comprising:
in a subject determined to have a decreased EKG-PR-interval, determining the amino acid present at position 646 of AKAP10/D-AKAP2 (SEQ ID NO:2) or the nucleotide present at position corresponding to nucleotide 2073 of SEQ ID NO: 1, wherein the presence of Val at position 646 of SEQ ID NO:2 or the presence of a -G- at nucleotide position 2073 of SEQ ID NO:1, indicates increased susceptibility to a disease or disorder.

54-58. (canceled)

59. A kit, comprising the combination of claim 57, further containing one or more components selected from the group consisting of a reagent for detecting a primer or probe that specifically hybridizes adjacent to or at a polymorphic region spanning a position corresponding to position 883 of SEQ ID NO 1 or 3, and a reagent for amplifying a primer specifically hybridizes adjacent to or at a polymorphic region spanning a position corresponding to position 883 of SEQ ID NO 1 or 3.

60. A method for indicating increased susceptibility of a subject to a disease or disorder, comprising:
conducting an EKG examination;
determining the EKG-PR-interval in the subject, wherein, if the EKG-PR-interval is decreased, then
determining the amino acid present in the subject at position 249 of AKAP10/D-AKAP2 (SEQ ID NO:2) or the nucleotide present at position corresponding to nucleotide 883 of SEQ ID NO: 1, wherein the presence of His at position 249 of SEQ ID NO:2 or the presence of a -A- at nucleotide position 883 of SEQ ID NO:1, indicates increased susceptibility to a disease or disorder.

61. A method for determining responsiveness of a subject to one or more β-blocking agents, comprising:
detecting for the subject the presence or absence of His at position 249 of SEQ ID NO:2 or a -A- nucleotide at a position corresponding to position 883 of SEQ ID NO: 1, wherein the presence of a His at position 249 of SEQ ID NO:2 or a -A- at nucleotide 883 of SEQ ID NO:1, is indicative of an increased likelihood that a subject has a modulated response to one or more β-blocking agents compared to a subject who does not have the allelic variant.

62. The method of claim 60, wherein the disease or disorder is selected from the group consisting of cardiovascular disorders, cardiac disease, proliferative disorders, neurological disorders, neurodegenerative disorders, obesity, diabetes and peripheral retinopathies.
